(19) United States
(12) Patent Application Publication
(10) Pub. No.: US 2023/0129111 A1
(43) Pub. Date: Apr. 27, 2023

(54) SYSTEM AND METHODS FOR FILLING ACCOMMODATING INTRAOCULAR LENSES WITH LIQUID

(71) Applicant: ForSight Vision6, Inc., South San Francisco, CA (US)

(72) Inventors: Guy Oren, South San Francisco, CA (US); Matthew Clarke, South San Francisco, CA (US); Nicole Kahn-Dror, South San Francisco, CA (US); Rina Tsvet, South San Francisco, CA (US); Bar Ovadia, South San Francisco, CA (US)

(21) Appl. No.: 17/970,131

(22) Filed: Oct. 20, 2022

Related U.S. Application Data
(60) Provisional application No. 63/400,622, filed on Aug. 24, 2022.
(60) Provisional application No. 63/270,252, filed on Oct. 21, 2021.

(51) Int. Cl. B29D 11/02; A61F 2/16

(52) U.S. Cl. CPC B29D 11/023; A61F 2/1635; A61F 2240/001; A61F 2250/0003

(57) ABSTRACT

A filling apparatus for filling a lens device with a volume of optical liquid including a dispensing system, a venting system having a vacuum pump and a vacuum chamber, a measurement system, and a lens device holding system. The dispensing system includes a source of optical liquid, a positive displacement pump, and a filling needle having a lumen in fluid communication with the source of optical liquid. The filling needle is configured to penetrate an injection zone of the lens device for filling an internal chamber of the lens device. The measurement system is configured to measure a lens zone of the lens device. The lens device holding system includes a lens fixture for maintaining a position of the lens device relative to the filling needle and the measurement system. Related systems, devices, and methods are provided.

### CROSS-REFERENCE TO RELATED APPLICATIONS

This application claims the benefit of priority under 35 U.S.C. § 119(e) to Provisional Patent Application Serial Nos. 63/270,252 filed Oct. 21, 2021, and 63/400,622 filed Aug. 24, 2022. The disclosures of the provisional applications are incorporated by reference in their entireties.

### BACKGROUND

A healthy, young human eye can focus an object in far or near distance, as required. The capability of the eye to change back and forth from near vision to far vision is called accommodation. Accommodation occurs when the ciliary muscle contracts to thereby release the resting zonular tension on the equatorial region of the capsular bag. The release of zonular tension allows the inherent elasticity of the lens to alter to a more globular or spherical shape, with increased surface curvatures of both the anterior and posterior lenticular surfaces.

The human lens can be afflicted with one or more disorders that degrade its functioning in the vision system. A common lens disorder is a cataract which is the opacification of the normally clear, natural crystalline lens matrix. The opacification can result from the aging process but can also be caused by heredity or diabetes. In a cataract procedure, the patient's opaque crystalline lens is replaced with a clear lens implant or IOL.

An artificial intraocular lens (IOL) are typically implanted after cataract extractions. Generally, IOLs are made of a foldable material, such as silicone or acrylics, for minimizing the incision size and improving patient recovery time. Most commonly used IOLs are single-element lenses that provide a single focal distance for distance vision. Accommodating intraocular lenses (AIOLs) have also been developed to provide adjustable focal distances (or accommodations) that rely on the natural focusing ability of the eye, for example, as described in US 2009/0234449, US 2009/0292355, US 2012/0253459, U.S. Pat. No. 10,258,805, and US 2019/0269500, which are each incorporated by reference herein in their entireties. AIOLs are beneficial for patients not suffering from cataracts, but who wish to reduce their dependency on glasses and contacts to correct their myopia, hyperopia and presbyopia. Intraocular lenses used to correct large errors in myopic, hyperopic, and astigmatic eye are called “phakic intraocular lenses” and are implanted without removing the crystalline lens. In some cases, aphakic IOLs (not phakic IOLs) are implanted via lens extraction and replacement surgery even if no cataract exists. During this surgery, the crystalline lens is extracted and an IOL replaces it in a process that is very similar to cataract surgery. Refractive lens exchange, like cataract surgery, involves lens replacement, requires making a small incision in the eye for lens insertion, use of local anesthesia and lasts approximately 30 minutes.

IOLs, particularly AIOLs, may incorporate liquids in fluid chambers such that accommodation is achieved with the help of fluid-actuated mechanisms. A force exerted on a portion of the lens is transmitted via the fluid to deform a flexible layer of the lens resulting in accommodative shape change of the AIOL. For example, ciliary muscle movements of the eye may be harnessed by components of an AIOL to drive shape change and accommodation. The AIOLs can achieve an optical power or diopter (D) in a desired range due to shape change of the optic upon application of a small amount of force (e.g., as little as 0.1-1.0 grams force (gf)) applied by the eye tissue. The AIOLs provide reliable dioptric change by harnessing small forces. A chamber for containing liquid materials that is formed by flexible layers of elastomeric material can change shape and thus, power of the lens depending on the volume of liquid. As fill volume increases beyond the chamber volume, the flexible layers can bulge outward creating a lens with a greater focal length.

There is need in the art for improved filling systems for flexible, shape-changing lenses to ensure precise volumes are delivered to achieve desired optical power for patients in need. The disclosure is directed to this, as well as other, important ends.

### SUMMARY

In an aspect, provided is a filling apparatus for filling a lens device with a volume of optical liquid including a dispensing system, a venting system, a measurement system, and a lens device holding system. The dispensing system includes a source of optical liquid, a dispenser, and a filling needle having a lumen in fluid communication with the source of optical liquid. The filling needle is configured to penetrate an injection zone of the lens device for filling an internal chamber of the lens device with the volume of optical liquid. The venting system includes a vacuum pump and a vacuum chamber configured to be in fluid communication with the vacuum pump. The measurement system is configured to measure a lens zone of the lens device. The lens device holding system includes a lens fixture for maintaining a position of the lens device relative to the filling needle and the measurement system.

The vacuum pump can be configured to remove air from within the internal chamber during filling with the filling needle. The vacuum pump can be fluidly coupled to a proximal opening of a vent needle having a distal end configured to penetrate a region of the injection zone of the lens device so that a distal opening of the vent needle is positioned inside the internal chamber of the lens device. The vent needle can be coupled to a gantry system to achieve desired spatial orientation relative to the lens device. The vacuum chamber can be sized to contain at least the lens fixture. The vacuum pump can create a vacuum inside the vacuum chamber so that the lens device is contained within a negative pressure environment. The internal chamber of the lens device and the vacuum chamber can be at the same pressure during filling. The vacuum chamber can be sized to contain the lens fixture and at least a portion of the dispensing system.

The filling needle can include a beveled tip surrounding a distal opening from the dispense lumen. The filling needle can have a bevel angle between 10 degrees and 60 degrees. The filling needle can have a bevel length that is between 0.1 mm and 1.3 mm. The filling apparatus can further include a gantry system coupled to the filling needle. The gantry system can be configured to movable position the needle in X, Y, and Z spatial orientation. The gantry system can be configured to advance the filling needle through a lateral side of the lens device within the injection zone parallel to a horizontal plane of the lens device to position the distal opening entirely within the internal chamber of the lens device to be filled by the volume of optical liquid. The dispensing system can further include a needle valve located between the filling needle and the source of optical liquid. The needle valve can be pneumatically triggered to dispense the volume of optical liquid through the filling needle into the internal chamber.

The measurement system can be configured to measure a radius of curvature of the lens zone. The measurement system can be configured to measure lens power of the lens zone. The measurement system can be configured to measure the lens zone sagitta. The measurement system can include a non-contact probe positioned on one or more movable stages. The non-contact probe can be a chromatic confocal sensor. The non-contact probe can be a Shack-Hartmann wavefront sensor. The measurement system can be configured to monitor the lens zone of the lens device simultaneously and in real-time during filling of the internal chamber with the filling needle.

The lens fixture can include a recess and a central aperture. The recess can be sized and shaped to receive the lens device so that the lens zone of the lens device is positioned over the central aperture. The lens fixture can be made of metal, glass, and/or plastic material. The filling apparatus can further include a heating element configured to heat the optical liquid prior to entering the lens device. The dispenser can additionally include a heating element configured to heat the source of optical liquid. The dispenser can be a positive displacement pump. The dispensing system can further include a needle valve connected to the dispenser and to the source of optical liquid. The needle valve can be arranged to control flow of optical liquid into the chamber. The needle valve in a closed configuration can prevent the optical liquid from flowing through the filling needle and allow for vacuum to build in the vacuum chamber. The dispenser can be pneumatically triggered to dispense an amount of optical liquid from the source of optical liquid upon opening of the needle valve.

In an interrelated aspect, provided is a method of delivering an amount of optical liquid into a sealed compartment of an ophthalmic lens device to achieve a refractive power of the ophthalmic device for a patient in need of the ophthalmic lens device while removing residual air from the sealed compartment of the ophthalmic lens device.

The ophthalmic lens device can be an accommodating intraocular lens. The ophthalmic lens device can include a lens zone and an injection zone outside of the lens zone. The injection zone can be formed of a material configured to withstand puncturing with a needle and reseal upon removal of the needle. The lens zone can be located within an optical zone of the ophthalmic lens device and can be configured to undergo a shape change for accommodation of the ophthalmic lens device. The method can further include assessing in real-time a curvature of the lens zone without contact against the ophthalmic lens device during delivering the amount of the optical liquid. The method can further include adjust the delivering based on the curvature. Removing residual air can include drawing a vacuum through a vent needle where a distal opening of the vent needle is positioned within the sealed compartment. The ophthalmic lens device can be contained within a vacuum chamber to remove the residual air from the sealed compartment through the vent needle. The vacuum chamber and the vent needle can maintain the ophthalmic lens device in a pressure-neutral environment during delivering.

In some variations, one or more of the following can optionally be included in any feasible combination in the above methods, apparatus, devices, and systems. More details are set forth in the accompanying drawings and the description below. Other features and advantages will be apparent from the description and drawings.

### BRIEF DESCRIPTION OF THE DRAWINGS

These and other aspects will now be described in detail with reference to the following drawings. Generally, the figures are exemplary and are not to scale in absolute terms or comparatively but are intended to be illustrative. Relative placement of features and elements is modified for the purpose of illustrative clarity.

FIG. 1A is a box diagram illustrating an implementation of a filling apparatus for use with a lens device;

FIG. 1B is a box diagram illustrating the filling apparatus of FIG. 1A incorporating an electronics module;

FIG. 2A illustrates a perspective view of an implementation of a lens device having an internal chamber configured to receive a liquid dispensed using the filling apparatus of FIG. 1A; and

FIG. 2B is a cross-sectional view of the lens of FIG. 2A taken along arrow B-B;

FIG. 3A is an implementation of a filling apparatus;

FIG. 3B is a detailed view of the filling apparatus of FIG. 3A;

FIG. 4A is a cross-sectional view of a dispensing needle penetrating an injection zone of a lens device held within a lens fixture;

FIG. 4B is a detailed view of FIG. 4A;

FIG. 4C is a cross-sectional view of the dispensing needle after rotation relative to the injection zone of the lens device;

FIG. 4D is a detailed view of FIG. 4C;

FIG. 5 is a schematic view of a measurement system of a filling apparatus positioned relative to the lens device as it is filled with the dispensing system;

FIG. 6A is an interrelated implementation of a filling apparatus for use with a lens device;

FIG. 6B is a detailed view of the filling apparatus of FIG. 6A;

FIG. 7A is an interrelated implementation of a filling apparatus for use with a lens device incorporating a needle valve system for dispensing;

FIG. 7B is a detailed view of the filling apparatus of FIG. 7A taken at circle B-B;

FIG. 7C is a top plan view of the filling needle and the venting needle of FIG. 7B penetrating the lens device;

FIG. 7D is a detailed view of FIG. 7C showing injection zones of the lens device;

FIG. 7E is a top plan view of the filling needle penetrating the lens device;

FIG. 7F is a perspective, cut-away view showing the filling needle entering the fluid chamber of the lens device;

FIG. 8A is a schematic of the needle valve system of FIG. 7A;

FIG. 8B is a schematic of the needle valve of FIG. 8A;

FIG. 9 is an interrelated implementation of a filling apparatus for use with a lens device;

FIG. 10A shows an interrelated implementation of a filling apparatus for use with a lens device in a loading configuration;

FIG. 10B is a top plan view of a holder for a lens device coupled to a filling needle of the filling apparatus of FIG. 10A;

FIG. 10C is a perspective view of the holder of FIG. 10B inserted within a cuvette for measurement under an evaluation beam.

It should be appreciated that the drawings are for example only and are not meant to be to scale. It is to be understood that devices described herein may include features not necessarily depicted in each figure.

### DETAILED DESCRIPTION

Ophthalmic devices, such as accommodating intraocular lenses (AIOLs) are typically made from soft flexible materials (e.g., silicone, acrylic, urethane) and can incorporate an internal chamber or reservoir for containing an optical liquid, such as silicone oil. As the liquid is injected to fill the chamber of the lens the flexible material of the lens body expands or is urged outward to a desired radius of curvature that determines the power of the lens. Fill volume changes the optical refractive power of the lens. As fill volume increases, optical power also increases because the increased size or thickness of the lens body creates a greater radius of curvature. It is important for liquid-filled lenses to have accurate filling in order to create a lens with specific focal length (power D) as well as quality optics. Described herein are manufacturing techniques and filling systems for providing a precise amount of liquid into sealed compartments of ophthalmic devices.

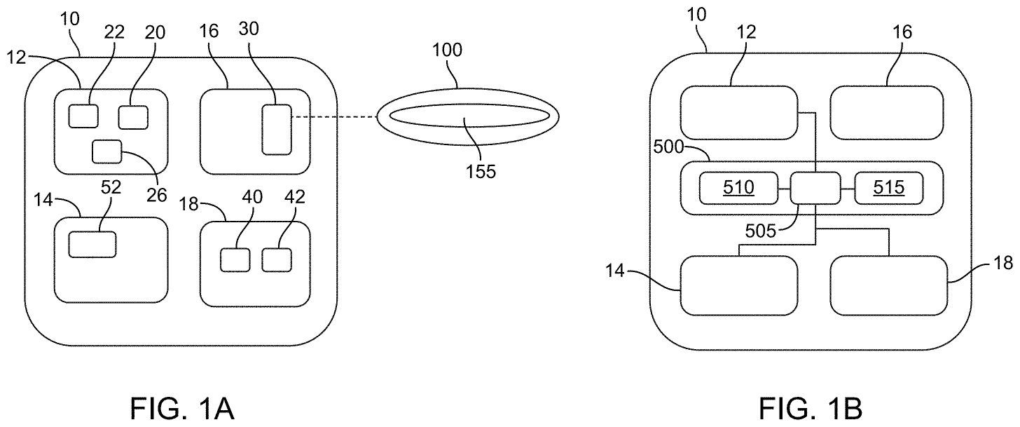

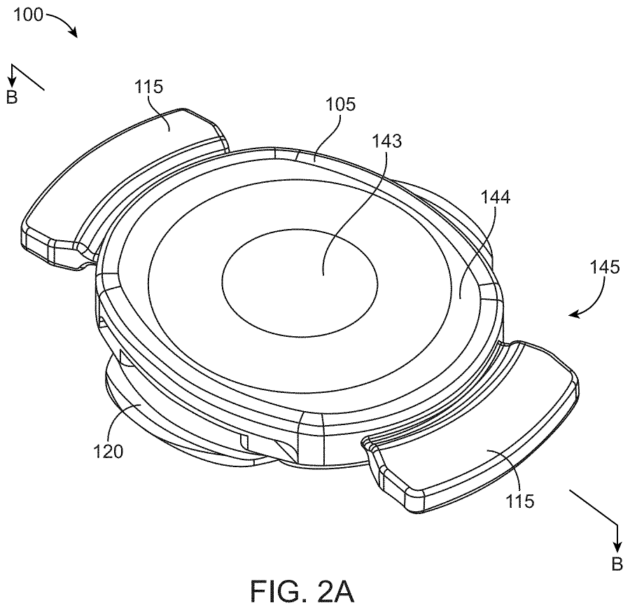

FIG. 1A is a box diagram of an implementation of a filling apparatus 10 designed specifically for use with thin ophthalmic devices 100, such as intraocular lenses. The apparatus 10 is configured to inject an appropriate volume of liquid optical material into the lens device 100 and adjusts the lens device 100 to the appropriate refractive power. The lens device 100 is preferably filled using the filling apparatus 10 during manufacturing of the lens device and prior to surgical implantation of the lens device. The filling apparatus 10 can include a dispensing system 12, a venting system 14, a lens device holding system 16, and a measurement system 18. The dispensing system 12 is configured to inject a volume of liquid optical material into the lens device 100, which can be held fixed by the lens device holding system 16. The venting system 14 is configured to remove residual air from the lens device 100 during filling with the dispensing system 12. The measurement system 18 is configured to assess, optionally in real-time, and without contact, the curvature of the lens zone of the lens device 10 during and/or upon filling to confirm accurate and precise volumes dispensed into the lens device by the dispensing system 12 to ensure a desired lens power is achieved. The measurement system 18 can be performed after filling or in real-time simultaneously with filling in order to adjust the filling to achieve the desired result. A user can complete the process without transferring an ophthalmic device 100 between holders of the holding system 16. The holding device configured to hold the ophthalmic device 100 during filling with the filling apparatus 10 may also be used to hold the ophthalmic device 100 during measurement and evaluation of filling. The holder may be inserted into a cuvette configured to be used with the measurement system directly in line with an evaluation beam as will be described in more detail below.

FIG. 1B is a box diagram of the filling apparatus 10 of FIG. 1A in which the various systems are configured to be in communication with one another via an electronics module 500, for example, to control the amount of oil dispensed into the lens device. The electronics module 500 can include a controller 510 in operative communication with one or more components of the apparatus 10 including the dispensing system 12, the venting system 14, and/or the measurement system 18. The systems can communicate information in real-time to the controller 510. The information can be displayed to the user via a user interface 505 for a user to then adjust or continue filling of the lens device 100. The systems can communication information to the controller 510 such that the controller 510 automatically adjusts to achieve a desired filling, venting, positioning, measuring, or other function of the apparatus 10.

The user interface 505 can receive manual input from a user and may include at least one actuator, trigger, pushbutton, keypad, touchscreen, or other input. The user interface 505 may include at least one light, screen, display or other visual indicator to provide instructions and/or information to the user, such as when to stop filling. The user interface 505 may include auditory, visual, or tactile indicators as well. For example, the user interface 505 can provide the user with alerts and information regarding the status of the apparatus 10 and systems during use such that manual and/or automatic adjustments can be made. The user interface 505 can include an LED or other type of display using, for example, electrical filaments, plasma, gas or the like. The user interface 505 can include a touch-screen type of display. It should be appreciated that the apparatus 10 need not include a user interface 505 or may incorporate more than a single user interface.

The controller 510 can include at least one processor and a memory device. The memory may be configured for receiving and storing user input data as well as data acquired during use of the apparatus 10, such as from the measurement system 18. The memory can be any type of memory capable of storing data and communicating that data to one or more other components of the device, such as the processor. The memory may be one or more of a Flash memory, SRAM, ROM, DRAM, RAM, EPROM, dynamic storage, and the like. The memory can be configured to store user information, history of use, measurements made, and the like.

The electronics module 500 can include a communication module 515 including a wired communication port, such as a RS232 connection, USB connection, Firewire connections, proprietary connections, or any other suitable type of hard-wired connection configured to receive and/or send information. The communication module 515 can alternatively or additionally include a wireless communication port such that information can be fed between the apparatus 10 via a wireless link, for example to display information in real-time. The wireless connection can use any suitable wireless system, such as Bluetooth, Wi-Fi, radio frequency, ZigBee communication protocols, infrared or cellular phone systems, and can also employ coding or authentication to verify the origin of the information received. The wireless connection can also be any of a variety of proprietary wireless connection protocols.

Each of the systems and devices will be described in more detail below.

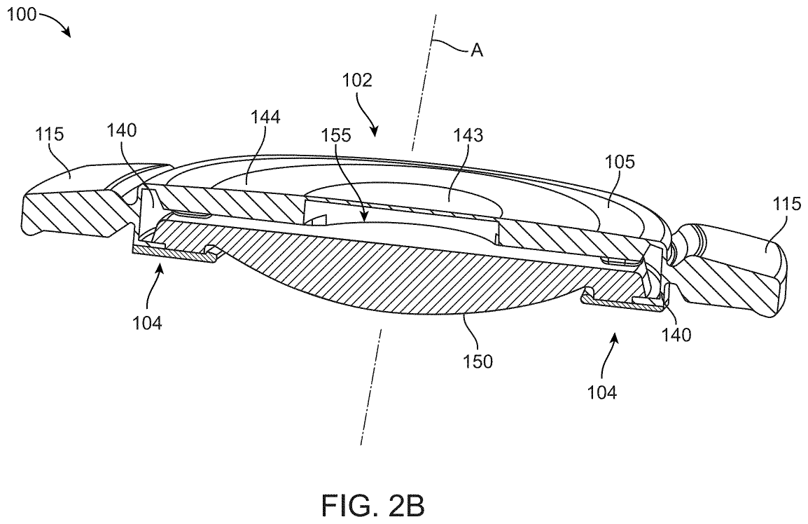

FIGS. 2A-2B illustrate an implementation of a thin ophthalmic device 100 that can be filled using the filling apparatus 10. FIG. 2A is a perspective view of the device 100 and FIG. 2B is a cross-sectional view of the device 100 showing the internal chamber designed to hold the volume of injected liquid optical material. The lens device 100 has solid optical component and liquid optical material. The liquid optical material is contained within a sealed, fixed volume fluid chamber 155 defined within the lens body 105 of the lens device 100. The lens device 100 is provided as an example only for clarity. Any of a variety of lenses including liquid optical material is considered herein for being filled using the filling apparatus, for example, lens devices described in US 2009/0234449, US 2009/0292355, US 2012/0253459, U.S. Pat. No. 10,258,805, and US 2019/0269500, which are each incorporated by reference herein in their entireties.

The solid optical component of the lens body 105 can include an optic configured to change shape or otherwise deform to change the shape of the lens body 105 for accommodation. The optic configured to change shape is shown herein as being on an anterior side of the lens body 105, but can form any of a variety of walls of the lens body 105. Relative orientations like “anterior” and “posterior” are provided for clarity sake in referring to the device and can vary. In some implementations, the anterior optic can have a central, dynamic zone or shape change membrane 143 surrounded by a static anterior optical portion 144 at a periphery of the anterior optic. The dynamic membrane 143 is configured to undergo a shape change or deformation whereas the static anterior optical portion 144 can be configured to resist or not to undergo a shape change. The static element 150, which can be a static lens, may not undergo a shape change as well. The cross-sectional geometry of the static anterior optical portion 144 and the dynamic membrane 143 can vary. Where the cross-sectional thickness of the membranes appear uniform in thickness in the figure it should be appreciated that the thickness may vary.

The lens devices 100 described herein are preferably formed of materials configured for small incision implantation. The solid optical components of the lens device 100 can have elastomeric characteristics and can be made of soft silicone polymers that are optically clear, biocompatible, and in certain circumstances flexible having a sufficiently low Young's modulus to allow for the lens body to change its degree of curvature during accommodation. It should be appreciated that some solid optical components have a different Young's modulus than other solid optical components to provide different function to the lens (e.g., outward bowing during accommodation compared to immovable static anterior optical portions mitigating distortion during accommodation). Suitable materials for the solid optical component of the lens can include, but are not limited to silicone (e.g., alkyl siloxanes, phenyl siloxanes, fluorinated siloxanes, combinations/copolymers thereof), acrylic (e.g., alkyl acrylates, fluoroacrylates, phenyl acrylate, combinations/copolymers thereof), urethanes, elastomers, plastics, combinations thereof, etc. In aspects, the solid optical component of the lens device 100 is formed of a silicone elastomer. The solid optical component can be formed of one or a combination of the materials described herein in which the liquid optical material described herein is fully encapsulated by the solid optical component. The solid optical component of a lens may include one or more regions that are configured to be in contact with and/or contain the liquid optical material. The liquid optical materials described herein can be specially formulated relative to the material of the solid optical component to mitigate lens instability and optimize optical quality. The liquid optical materials, sometimes referred to herein as an optical fluid, can include any of a variety of copolymers, including fluorosilicone copolymers and other liquid optical materials as described in PCT Application No. PCT/US2021/37354, filed Jun. 15, 2021, which is incorporated by reference herein in its entirety.

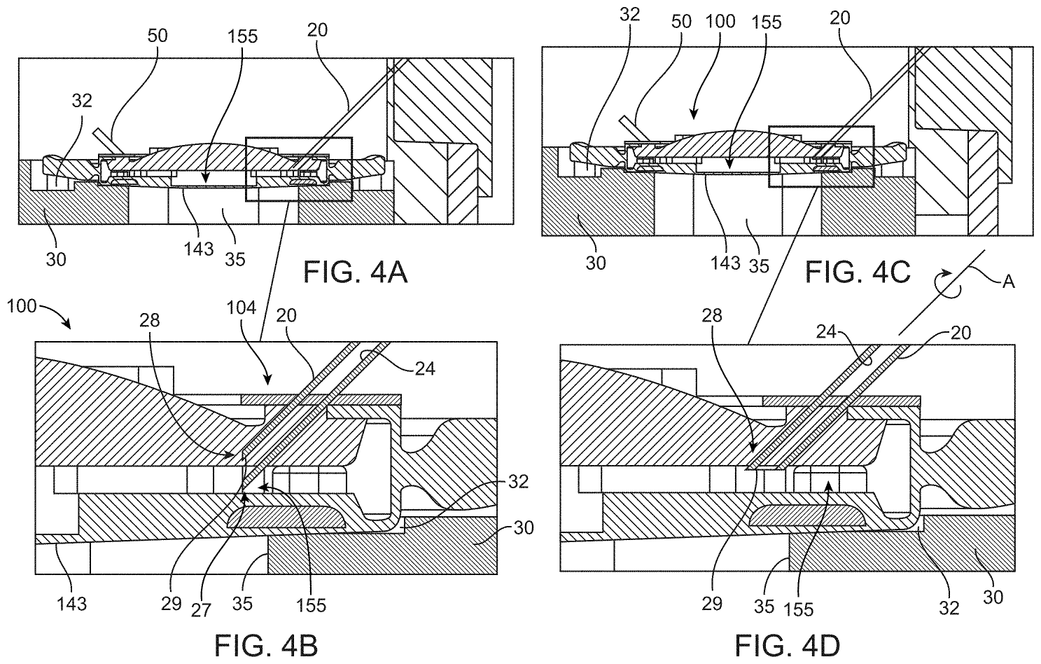

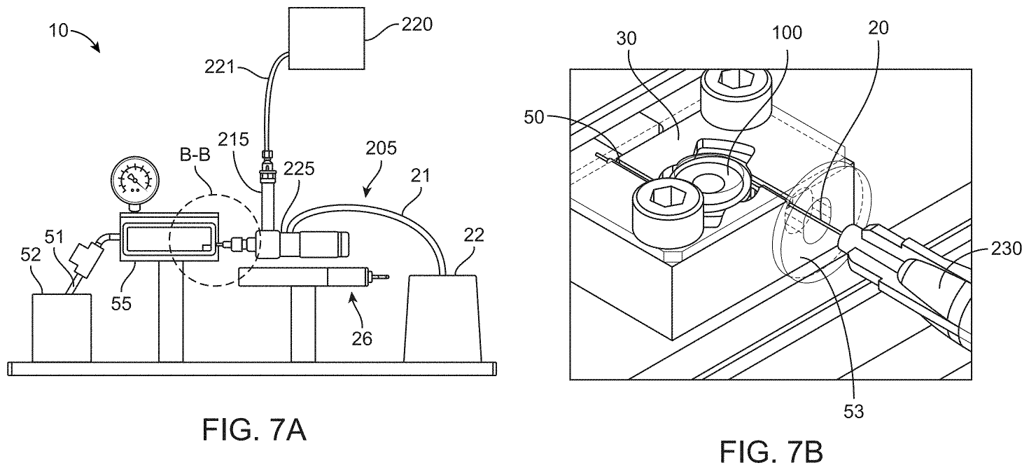

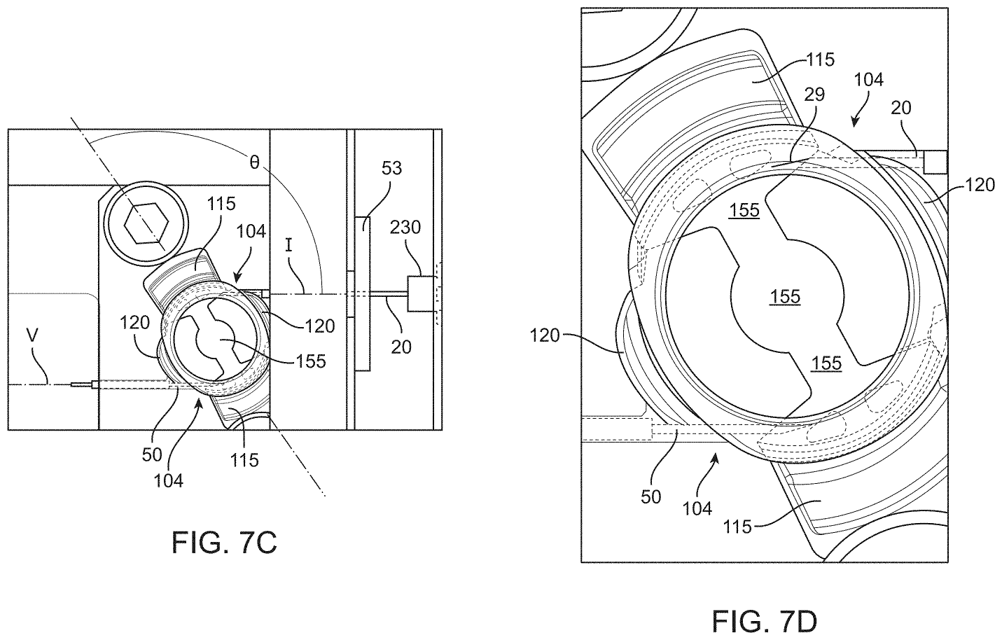

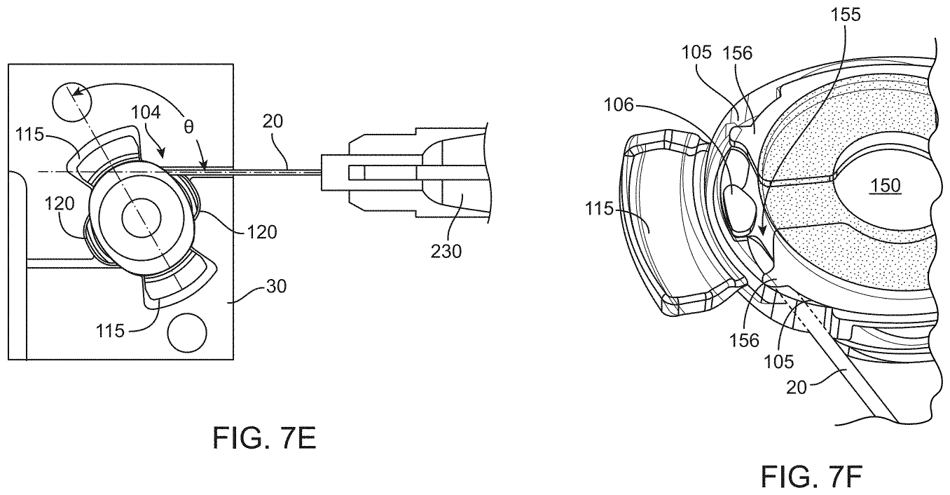

Again with respect to FIG. 2A, the lens device 100 can include a lens zone 102 and an injection zone 104, preferably located outside of the lens zone 102 near a perimeter of the device although this is not absolutely necessary. The lens zone 102 can include portions of the lens device 100 that are involved in accommodation and vision. The injection zone 104 can include portions of the lens device 100 that are configured to be penetrated by the filling apparatus 10 for injection of the liquid optical material into the lens device 100. The injection zone 104 can be located at any of a variety of locations on the lens device 100 that can withstand puncturing with a needle and reseal upon removal of the needle. In some implementations, the injection zone 104 can include one or more fill ports or sealable valves positioned on a posterior end region of the lens device 100 near the outer perimeter of the device (see FIGS. 4B, 4D). The filling needle 20 is shown penetrating the injection zone 104 at an angle relative to a horizontal plane of the lens device, for example, about 45 degrees from the horizontal plane of the lens device. In a preferred implementation shown in FIGS. 7A-7E, the injection zone 104 is located on a lateral side of the lens body. The filling needle 20 can penetrate the injection zone 104 at an angle that is substantially parallel to the horizontal plane of the lens device or about 180 degrees. The benefit of side filling is that the filling needle 20 can be a standard bevel angle and length without needing to be rotated in order to ensure the opening of the needle is within the sealed chamber. Needle rotation can pose a risk of displacing the lens from the holder during filling and thus, it is preferred to avoid any needle movement once the chamber is penetrated.

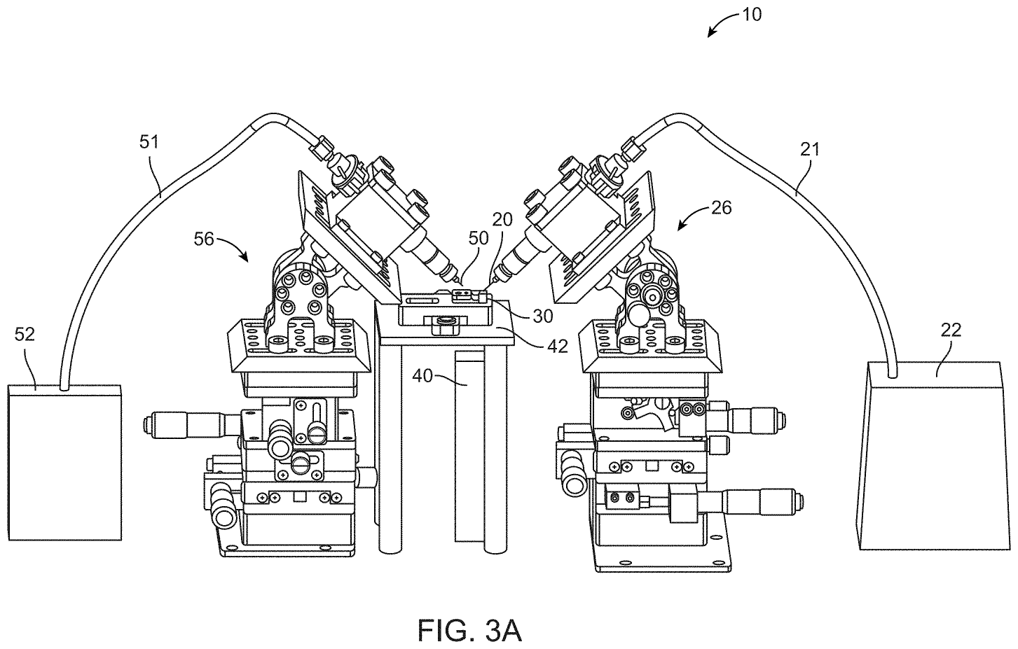

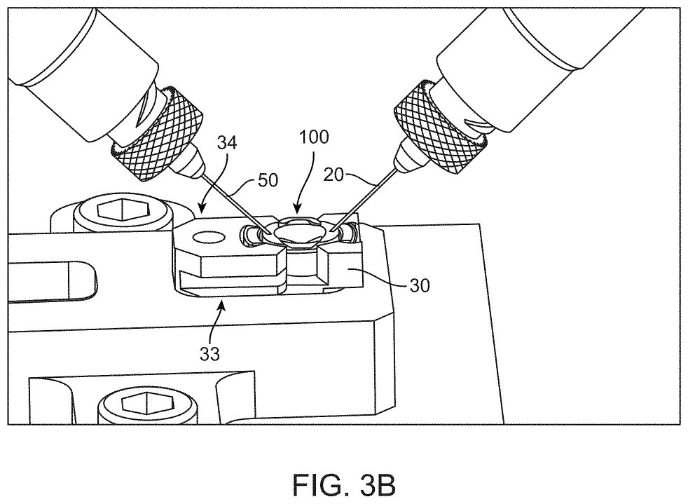

The injection zone 104 whether located on a posterior end of the lens device 100 or on a lateral side of the lens device 100 is preferably at a location that does not impact the movable parts of the lens, such as the force translation arms. The injection zone 104 of the embodiment in FIGS. 3A-3B is shown located within a quadrant between the location of the wing haptics and radially inward from the two force translation arms. The injection zone 104 of the embodiment in FIGS. 7A-7E is within a region that is between the force translation arm 115 and the wing haptic 120. The filling needle 20 can approach the injection zone 104 along axis I within the Z-plane (i.e., parallel to the horizontal plane of the lens device) and at an angle θ relative to the force translation arms 115 within the Z-plane that is between 110 and 130 degrees (see FIG. 7C). FIGS. 7E-7F show how the filling needle 20 can penetrate through both the lens body 105 and a non-optical portion of the posterior lens or static element 150 providing a longer path of silicone through which the needle inserts, which assists in sealing following removal of the needle. The non-optical portions of the posterior lens can be designed to receive the filling needle 20, such as by incorporating a peripheral feature 156 that abuts against a corresponding peripheral feature near a perimeter of the capsule 105 to guide the needle into the chamber 155 being filled (see FIG. 7F). A small bump or column 106 of silicone can be positioned distal to the sharp tip of the filling needle forming a sort of stop for the needle to discourage further penetration of the needle 20 relative to the lens. The angle of insertion (e.g., 120 degrees relative to the axis of the force translation arm 115) ensures the sharp tip of the needle 20 avoids contact with the shape deformation membrane 140, which could happen at larger angles, and avoids contact with the posterior lens 150, which could happen at smaller angles.

Again with respect to FIG. 7C, the filling needle 20 can penetrate the injection zone 104 on one quadrant of the lens device and the vent needle 50 penetrating the injection zone 104 on an opposite quadrant of the lens device. The filling needle 20 and vent needle 50 are shown parallel to one another along their respective axes (I and V axes shown in FIG. 7C) and parallel to a horizontal plane (Z-plane) of the device. It should be appreciated that the filling needle 20 and the vent needle need be advanced along axes that are non-parallel and can instead be advanced along axes that are at an angle relative to one another. The bevels of the needles 20, 50 penetrate a lateral side of the lens body through to a perimeter region of the fluid chamber 155 so that the distal opening 29 from the needles 20, 50 are positioned entirely within the chamber 155 without any rotation around the insertion axis. The distal end region of the needles 20, 50 can extend through the lens along a similar path as discussed above.

The lens device of FIGS. 2A-2B is illustrated as one of any of a variety of lens devices that can be filled using the apparatus described herein. The apparatus can be used to fill a lens device having solid lens material defining an internal chamber configured to contain a liquid lens material. At least a portion of the solid lens material forms an optic configured to move to change a shape and thus the power of the lens device. The lens device for use with the apparatus described herein can be an implantable lens device for positioning in the capsular bag, the ciliary sulcus, the anterior chamber, the posterior chamber, and/or the vitreal chamber. The lens device for use with the apparatus described herein can be a liquid lens that is not an implantable lens. For example, the lens device can be part of an endoscopic system for use inside a patient, but that is not implanted to stay inside the patient. The lens device need not be for internal use, but can be an external, wearable lens, such as a contact lens or a lens for eyeglasses. The lens device also need not be useful for treatment of a patient, but can include liquid lenses for other purposes, such as photography, microscopy, or other technology area in which precision optics is desirable.

FIG. 3A illustrates an implementation of the filling apparatus 10. FIG. 3B is a detailed view of the filling apparatus 10 of FIG. 3A. The filling apparatus 10 can include a dispensing system 12 having a filling needle 20 coupled to a fluid dispenser 22 via tubing 21. The fluid dispenser 22 can be a positive displacement pump configured to urge a volume of liquid from a source of the liquid through the tubing 21 and out a distal opening 29 of the filling needle 20. The pump can be a mechanical pump (e.g., gear, diaphragm, peristaltic, syringe), pneumatic pump, or other type of pump for dispensing small volumes of liquid. The fluid dispenser 22 of the dispensing system 12 can be a micro-computer controlled electronic-pneumatic system that dispenses the liquid into the lens device to reach a desired inflation level. The fluid dispenser 22 of the dispensing system 12 can be in communication with the measurement system 18 to control the amount of oil dispensed into the lens device based on the measured curvature or power of the lens device. The fluid dispenser 22 of the dispensing system 12 pushes air into the tubing to urge the oil from a syringe out through the filling needle 20. In other implementations, the fluid dispenser 22 of the dispensing system 12 can open a valve so that oil flows from the syringe, which is under constant pressure. For example, the dispenser 22 is pneumatically triggered to dispense an amount of oil from the syringe upon opening of an adjustable needle valve, which will be discussed in more detail below. The source of the liquid can be a container (not shown) that can hold a larger volume of the liquid so that smaller volumes of the liquid can be delivered through tubing 21 to the filling needle 20.

The liquid can be a non-compressible liquid or gel of high clarity and transmission in the visible spectrum, such as silicone oil. The silicone oil can vary depending on the material of the solid lens components. The fluid dispenser 22 can incorporate a heating element so that the liquid in the fluid dispenser 22 can be heated prior to injection into the lens device. Heating the liquid reduces liquid viscosity allowing for injection of the liquid through smaller gauge needles. In an implementation, the system can include a heating element configured to heat the optical liquid. The heating element can heat the liquid in the filling needle 20.

Still with respect to FIGS. 3A-3B, the filling needle 20 can be movably positioned relative to the lens device 100 manually or by a motorized precision gantry system 26 to achieve desired spatial orientation of the filling needle 20 relative to the lens device 100. The gantry system 26 can combine multiple axis of motion including rotation around a long axis of the needle 20 to create high precision positioning for the filling needle 20 relative to the lens device 100. The filling needle 20 is oriented relative to the lens device so as to penetrate a relatively thicker area of the lens device that can be penetrated without damaging the optical surfaces. The needle 20 is shown in FIG. 3B positioned at an approximately 45 degree angle relative to the lens device 100. The angle can vary between 90 degrees (perpendicular) to the lens device 100 up to 180 degrees (parallel) to the lens device 100 (see FIGS. 7A-7D). Filling from the side such that the angle of the filling needle 20 approaches 180 degrees or is parallel to a horizontal plane of the lens device 100 is preferred as it allows for the needle to be inserted into the chamber 155 without any needle rotation to orient the opening relative to the chamber. The opening of the filling needle 20 can be directly inserted into the chamber 155 without further manipulation beyond initial penetration along the horizontal plane of the lens device.

FIGS. 4A-4D are cross-sectional views of the filling needle 20 inserted through the lens device 100. The filling needle 20 can have a beveled tip 28 surrounding a distal opening 29 from a dispense lumen 24. The beveled tip 28 of the filling needle 20 is configured to penetrate the lens device 100 through the injection zone 104 while the lens device 100 is held fixed within the holding system 16. The lens device 100 can be relatively thin product (e.g., 0.5 mm-1.0 mm thick). The walls forming the internal chamber 155 can be even thinner (e.g., 0.2 mm-0.6 mm thick) resulting in a very thin internal chamber 155 (e.g., 0.1 mm-0.4 mm deep). In other implementations, the lens device 100 is configured to be positioned inside the capsular bag and may be sized to fill the bag. Such a lens device may have a larger internal chamber that is between 0.5 mm deep up to about 2.0 mm deep. The lens thickness between the anterior surface and the posterior surface can be up to about 2.5 mm. The walls forming the internal chamber 155 can be up to about 2.0 mm thick.

The size of the filling needle 20 as well as the length and angle of the beveled tip 28 are selected to ensure resealable penetration of the lens device 100 is achieved without inadvertent puncture of the lens device 100 outside of the injection zone 104. Standard thin needles have a relatively long bevel length due to a relatively shallow bevel angle. Bevel length (the distance between the heel of the needle bevel and the distal-most tip of the needle) can be calculated as the quotient of the outer diameter of the needle divided by the tangent of the bevel angle. As an example, a 30 G-32 G needle with a bevel angle of about 12 degrees can have a bevel length that is about 1.15 mm-1.5 mm. A 27 G needle having an outer diameter of about 0.413 with a point style 4 beveled at 30 degrees can have a bevel length that is 0.715 mm (0.413 mm/tan30). The filling needle 20 can be 32 G-34 G and have a bevel angle of between 25-50 degrees so that the bevel length is between 0.3 mm and 0.5 mm. The bevel length depends upon the needle diameter and bevel angle. The bevel length can vary between about 0.1 mm up to about 1.3 mm, preferably between 0.2 and 0.5 mm. It should be appreciated that the needle dimensions provided herein may vary depending on the type of needle bevel and whether more than a single slope defines the needle tip. In some implementations, where the lens device 100 is relatively thin (e.g., 0.2 mm-0.6 mm), the filling needle 20 can be inserted through the injection zone 104 a distance of at least about 300 microns or between 200-400 microns. For thicker lens devices this distance can be greater. Thus, the depth of penetration and distance the filling needle is inserted can vary depending on the thickness of the lens.

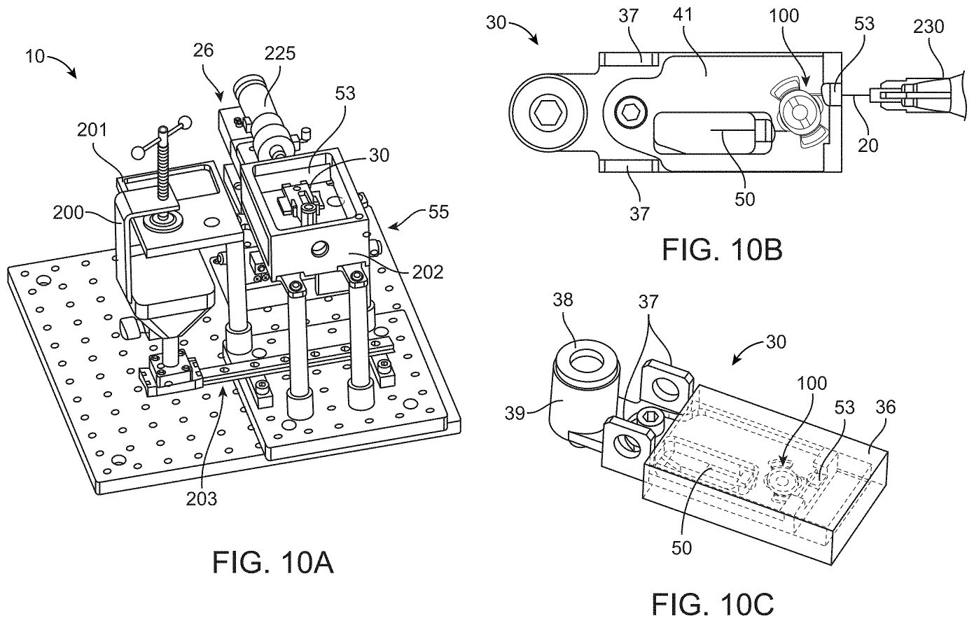

In preferred implementations, the lens device is filled from the side such that the filling needle is held substantially parallel to the horizontal plane of the lens device (i.e., about 180 degree angle) and the injection zone 104 is located on a lateral side of the perimeter of the device. In side filling systems, the bevel angle can be greater. Side filling systems, such as those shown in FIGS. 7A-7F and also FIGS. 10A-10C allow for the use of standard gauge filling needle having standard bevel angles and bevel lengths (e.g., 31 G needle having a 12-15 degree bevel angle). Side filling systems also avoid the need for rotating the filling needle around its axis after initial penetration and before injection as discussed below. The side filling system will be discuss in more detail below.

The gauge and bevel length of the needle(s) used to penetrate the lens device can vary depending on the size of the internal chamber 155 of the lens device 100 being filled. For example, a lens device that is larger or has a deeper internal chamber 155 can accommodate greater bevel lengths without risk of inadvertent penetration of the opposite wall. But it is generally desirable to limit the gauge of the needle to avoid coring the lens device and ensuring the chamber remains sealed following removal of the needle. The needle can range between 34G up to about 27G. Fluid flow through smaller needle gauges can be improved by heating the liquid being injected to reduce the liquid viscosity.

The beveled tip 28 can be pushed through the injection zone 104 by the gantry system 26 a distance along a longitudinal axis of the needle 20. The distance of insertion can vary depending on the dimensions of the lens device 100 being filled. In some implementations, the internal chamber is about 0.1 mm-0.4 mm deep and located approximately 0.2-0.4 mm from the external surface of the injection zone 104. The beveled tip 28 of the filling needle 20 can be advanced a distance of between 200 microns and 400 microns, or preferably about 300 microns. The distance the beveled tip 28 of the filling needle 20 penetrates the lens device 100 regardless the actual dimensions of the device is sufficient that at least a portion of the distal opening 29 enters the chamber 155 (see FIGS. 4A-4B). After penetration of the chamber 155, the filling needle 20 can be rotated by the gantry system 26 around the longitudinal axis and relative to the lens device 100, for example about 180 degrees, until the orientation of the beveled tip 28 ensures the distal opening 29 is entirely within the internal chamber 155 of the lens device 100 (see FIGS. 4C-4D). FIGS. 4A-4B show the filling needle 20 just after penetration of the injection zone 104. FIGS. 4C-4D show the filling needle 20 after rotation of the needle 20 relative to the lens device 100. The distal-most tip 27 of the needle 20 is prevented from contacting the opposite side of the lens device 100 to prevent inadvertent damage. Rotation of the filling needle in order to place the needle opening within the internal chamber being filled may be an unnecessary step for thicker lenses. The rotation of the filling needle allows for relatively thin lenses with insufficient space for the entire bevel to receive the needle for filling.

The holding system 16 is designed to maintain a position of the lens device 100 relative to the dispensing system 12 and the measurement system 18. The lens device 100 need not be transferred between different holding systems so that a single holding system 16 can be used with both the dispensing system 12 and the measurement system 18. In some implementations, the filling needle 20 of the dispensing system 12 can penetrate the injection zone 104 of the lens device 100 being held by the holding system 16 while the measurement system 18 simultaneously monitors the lens zone 102. Meaning, both the lens zone 102 and the injection zone 104 of the lens device 100 remain accessible within the holding system 16 during use of the filling apparatus 10.

The holding system 16 can include a lens fixture 30 having a recess 32 sized and shaped to receive at least a portion of the lens device 100. FIG. 3B illustrates the lens device 100 sitting within the recess 32 of the lens fixture 30 of the holding system 16. FIGS. 4A-4D are cross-sectional views of the lens device 100 sitting within the recess 32 of the fixture 30.

The lens device 100 can be inverted within the fixture 30 so that the lens zone 102, which in this lens device 100 is on an anterior region of the device, is directed toward a lower end 33 of the fixture 30 and the injection zone 104 of the lens device 100, which in this lens device 100 is on a posterior region of the device, is directed toward an upper end 34 of the fixture 30. Although the lens device 100 is shown oriented within the lens fixture 30 in an inverted position, the lens device 100 can be oriented within the lens fixture 30 or relative to the apparatus 10 in any of a variety of orientations, including vertical or horizontal, anterior position up or down. Inverting the lens device 100 within the fixture 30 can allow gravity to assist in the egress of air from the internal chamber 155, which will be described in more detail below. The internal chamber 155 of the lens device 100 may have an irregular shape such that air tends to remain inside despite the volume of liquid dispensed. Inverting the lens device 100 and thus, the internal chamber 155 means that dispensing liquid from the lower region of the internal chamber 155 (or an upper region once inverted) uses gravity to urge the air out from the internal chamber 155 upon filling.

The lens fixture 30, which can be made of metal, glass, plastic, or other relatively rigid material, can include a central aperture 35 extending from the lower end 33 through to the upper end 34 of the fixture 30. The central aperture 35 allows for an evaluation beam to access the lens device 100 held by the fixture 30 and avoids needing to transfer the lens device 100 from the lens fixture 30 for measurement purposes. The fixture can have a generally annular shape for receiving the lens device 100. The recess 32 can be sized and shape to receive the lens device 100 so that the lens zone 102 of the lens device 100 is positioned over the central aperture 35. The central aperture 35 prevents the fixture 30 from coming into contact with or covering the central lens zone 102 of the lens device 100 when the lens device 100 is positioned within the recess 32. The fixture 30 can support the outer perimeter region of the lens device 100, particularly the injection zone 104, so that it aids in supporting the device during penetration with the filling needle 20. The central aperture 35 also allows for the measurement system 18 to be directed toward the lens zone 102, for example, allowing for monitoring of filling, whether in real-time or in stages.

FIGS. 10B-10C show a fixture 30 holding a lens device 100. FIG. 10B shows the fixture 30 holding the lens device 100. FIG. 10C shows the fixture 30 holding a lens device 100 positioned within a cuvette 36. The fixture 30 allows for a lens device 100 to be filled and measured so as to complete the process without transferring the lens device 100 between different fixtures. The fixture 30 can include gripping arms 37 or other feature configured to allow a user to easily hold the fixture 30. The gripping arms 37 can be positioned away from the location of the lens device 100 so as to avoid a user coming into contact with the lens device 100 positioned within the fixture 30. The fixture 30 can include a coupler 38 that allows for it to be removably affixed to the base. The coupler 38 can be attached to the fixture 30 in a manner that avoids the coupler 38 coming fully detached from the fixture 30 even when detached from the base. The coupler 38 is shown positioned within a housing 39 that prevents the coupler 38 from coming out of the housing 39. The lens device 100 can be held within the recess 32 of the fixture 30 by a lid 41 that is configured to be positioned over the lens device 100. The lid 41 can be attached to the fixture 30 such as with a screw or other coupler. At least a portion of the fixture 30 near the lens device 100 held within the recess 32 can have a minimized thickness (e.g., less than about 5 mm thick) sized to insert within a cuvette 36 (see FIG. 10C). The cuvette 36 provide proper orientation relative to the evaluation beam of the measurement system. As discussed elsewhere herein, the lens zone 102 of the lens device 100 can include a dynamic membrane 143 made from a soft elastomeric material (e.g., 50-150 microns thick of a 20-60 ShoreA material). Prior to filling the internal chamber 155, the dynamic membrane 143 may be in a substantially pressure-neutral position to ensure the membrane is kept generally planar and does not deform during air removal. The pressure-neutral position can be achieved by evacuating the environment internal to the membrane (i.e., the internal chamber 155) and evacuating the environment external to the membrane (i.e., pressure chamber space within which the lens device sits) so that the pressures in environments equilibrate prior to dispensing the liquid into the chamber 155. Once the air is removed, liquid from the dispenser 22 can be injected through the filling needle 20. As the liquid from the dispenser 22 is injected through the filling needle 20 into the internal chamber 155, the dynamic membrane 143 may bow outward to take on a convex lens shape. Fluid can be injected and/or withdrawn until the desired curvature or power of the lens zone 102 is achieved. The measurement system 18 of the filling apparatus 10 is configured to assess the lens device 100, which can be performed in real-time during filling, for example, by measuring the curvature or power of the lens device 100. The system need not perform simultaneous real-time monitoring and measurement with the measurement system 18 during filling, but instead can rely on controlled oil delivery in which each diopter of the lens device is equal to 0.0001 gr oil. The system can also rely on the assistance of external power measurement or radius measurement devices, but not necessarily in real-time. Instead, the lens device 100 held within the lens holder can be transferred between the filling apparatus 10 and the measurement system 18 to confirm and monitor the curvature or power of the lens during 100 in stages.

The amount of liquid injected into the internal chamber can be adjusted for the volume of the needle(s) positioned within the internal chamber 155 during filling. The volume can vary depending on needle size and insertion depth of the needle. The insertion depth of the needle during filling can vary depending on the thickness of the lens device. For a relatively thin lens device, the depth of penetration of the needle during injection of the liquid is preferably minimized (see, e.g., FIG. 4D). As an example, a 27 gauge needle can have a nominal OD of about 0.413 mm and can be inserted within an internal chamber by only about 0.1 mm. The volume of the internal chamber taken up by the needle during filling in this example is about 0.013 mm³. The internal chamber 155 can be overfilled by this amount to account for the reduction in volume that occurs upon withdrawal of the needle after filling such that a desired optical power or curvature is achieved.

The apparatus 10 can incorporate a viewing system, such as a microscope arranged so that a user can see the depth of penetration of the needle (filling needle and/or venting needle) into the lens device 100. In some implementations, the outside surface of the needle(s) can incorporate one or more markers that provide information regarding depth of penetration of the needle through the lens device. Upon reaching a particular marker on the needle observed by microscopic viewing, a user can determine a displaced volume and the filling can be adjusted based on this displaced volume so that desired optical power or curvature is achieved even upon removal of the needle(s). In other implementations, depth of penetration of the needle can be assessed based on a first sensor, such as an axial force gauge, that senses when the needle first comes into contact with the lens device and a second sensor, such as an axial motion sensor, that measures axial distance moved beyond that point further into the lens device.

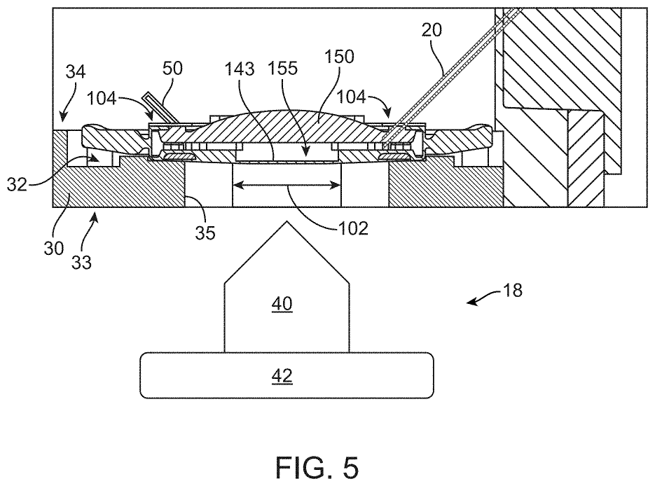

The measurement system 18 can include a non-contact probe 40 (see FIG. 5). The probe 40 can be a chromatic confocal sensor capable of measuring the radius of curvature of the lens zone 102 of the dynamic membrane 143. The probe 40 can be positioned on one or more stages 42 configured move the probe 40 in x, y, and z directions relative to the lens device 100 held within the lens fixture 30. The measurement system 18 can include two linear stages—one stage moving in the x and y directions and a second stage moving in the z direction. The probe 40 can scan across 2 perpendicular axes of the lens device 100 through the lens center upon request. The radius of curvature can be calculated by the measurement system 18 and compared to a desired radius based on the desired lens curvature. The difference in volume can be calculated and an appropriate amount of liquid injected into the lens device 100. The probe 40 of the measurement system 18 can alternatively be configured to determine the power of the lens device 100 during filling with the dispensing system 12 (in real-time or in stages) as the basis for determining when the lens device 100 has be filled to the correct degree. For example, the probe 40 can be a Shack-Hartmann wavefront sensor or other wavefront aberrometry system, OCT, or optical imaging system. In still further implementations, the height of the lens device 100 or sagitta of the lens zone can be monitored, for example, from a 90 degree angle. The sagittal depth of the lens can be measured and serve as a surrogate for measuring the curvature. The measurement system 18 can be configured to provide optical imagery feedback and incorporate suitable instrumentation, such as an aberrometer, a fractometer, or an imaging system to monitor the geometry and calculate the power of the lens device, such as an OCT system. To determine appropriate power, an OCT may be used to monitor the profile of the lens so that the lens geometry can be adjusted to the appropriate power and/or geometry. The measurement system 18 can provide this monitoring, in real-time or in stages, while the lens device 100 is in a vacuum environment on both sides of the optical element.

The filling apparatus 10 can also include a venting system 14. The venting system 14 allows for any air trapped inside the internal chamber 155 of the lens device 100 or any air bubbles within the liquid being dispensed to be removed to make space for the liquid during filling. The venting system 14 can include a separate vent needle 50 in fluid communication with a vacuum pump 52 via tubing 51 that is configured to draw air out of the lens device 100. The vacuum pump 52 can be any of a variety of pump configured to draw a fluid through the vent needle 50. The vent needle 50 can be movably positioned relative to the lens device 100 by a second precision gantry system 56 to achieve desired spatial orientation. Like the gantry system 26 for the filling needle 20, the gantry system 56 for the vent needle 50 combines multiple axes of motion to create high precision positioning for the vent needle 50 relative to the lens device 100.

The vent needle 50 can have a beveled tip surrounding a distal opening from the vent lumen. The beveled tip of the vent needle 50 is configured to penetrate the lens device 100 through another region of the injection zone 104 than the filling needle 20 while the lens device 100 is held fixed within the holding system 16. The size of the vent needle 50 as well as the length and angle of the beveled tip are selected to ensure resealable penetration of the lens device 100 is achieved without inadvertent puncture of the lens device 100 outside of the injection zone 104. The vent needle 50 can be the same size as the filling needle 20, for example, 32 G-34 G and have a bevel angle of between 10-60 degrees, or between 25-50 degrees, although the vent needle size can be smaller or larger than the filling needle size. The vent needle 50 can be rotated by the gantry system 56 after insertion through the injection zone 104 so that the distal opening is entirely within the internal chamber 155 of the lens device 100.

In use, the vent needle 50 can be inserted through a first region of the injection zone 104 of the lens device 100. The vent needle 50 can be inserted a distance (e.g., about 300 microns) and rotated about 180 degrees relative to the lens device 100 to ensure the distal opening from the vent needle 50 is fully positioned within the internal chamber 155. The filling needle 20 can then be inserted through a second region of the injection zone 104 of the lens device 100. The beveled tip 28 of the filling needle 20 can also be advanced and rotated as described to ensure the distal opening 29 from the filling needle 20 is positioned fully within the internal chamber 155. A vacuum can be drawn through the vent needle 50 via the vacuum pump 52 to draw any air from the internal chamber 155. The vacuum pump 52 can be turned off and the fluid dispenser 22 turned on to inject an amount of liquid to fill the internal chamber 155 to a desired curvature or power of the lens device 100. The lens curvature or power can be measured, in real-time or in stages, by the measuring system 18 to determine the ideal filling level.

In some implementations, the venting system 14 does not include a separate needle from the filling needle 20. Rather, the filling needle 20 can include more than a single lumen in order to both dispense and vent. The filling needle 20 can include the dispense lumen and a vent lumen. The dispense lumen and vent lumen can be concentric, coaxial lumens so that the lens device 100 is penetrated just a single time for filling and venting. Alternatively, the dispensing and venting can be performed through a single needle 20 having a Y-connection with a switching valve that allows it to be used for both venting and dispensing. In still further implementations, the venting system 14 has no venting lumen at all (whether in the same or different needle as the dispensing needle). Vacuum can be performed in the environment surrounding the lens device 100 so that air from within the internal chamber 155 permeates the silicone of the lens device 100. A slow vacuum process can be employed to slowly remove the air from the chamber 155 simply through the porosity of the silicone material of the lens device 100. Air movement through the dispensed liquid can be facilitated by heating, for example, by heating the lens fixture 30 within which the lens device 100 is held. The dispensed liquid can be heated within the fill needle by a heating element of the apparatus 10.

Placing the entire lens device 100 under vacuum conditions can improve filling and operation of the lens device 100 and prevent inadvertent damage of the soft, elastomeric components of the lens device 100. As discussed above, the vacuum pump 52 can be used to pump air directly from the internal chamber 155. This can cause the elastomeric dynamic membrane 143 to stretch, which can block removal of the air from all parts of the lens device 100. A dynamic membrane 143 that stretches beyond its elastic range can irreversibly damage the lens device and result in poor optical quality upon filling. Filling of the chamber 155 while the entire lens device 100 is under vacuum conditions can prevent this.

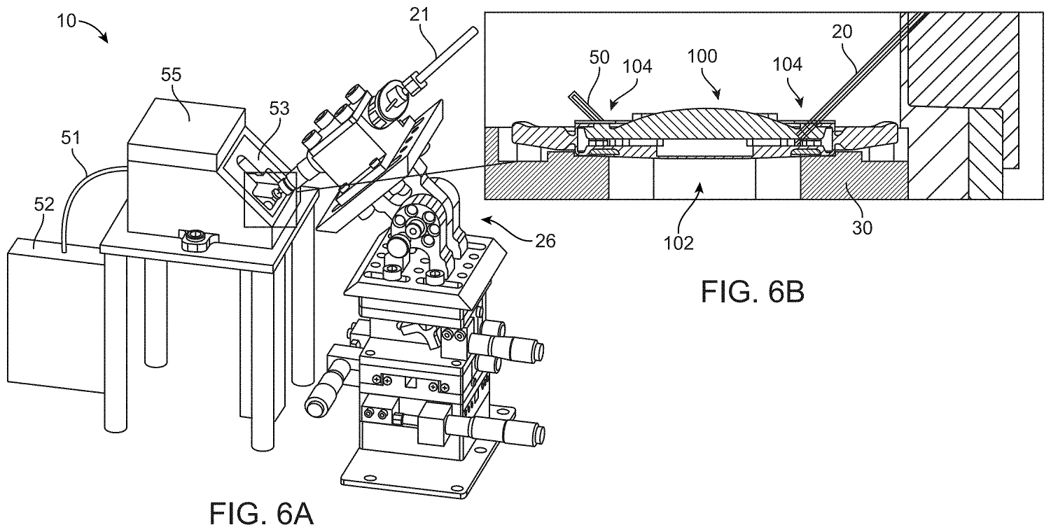

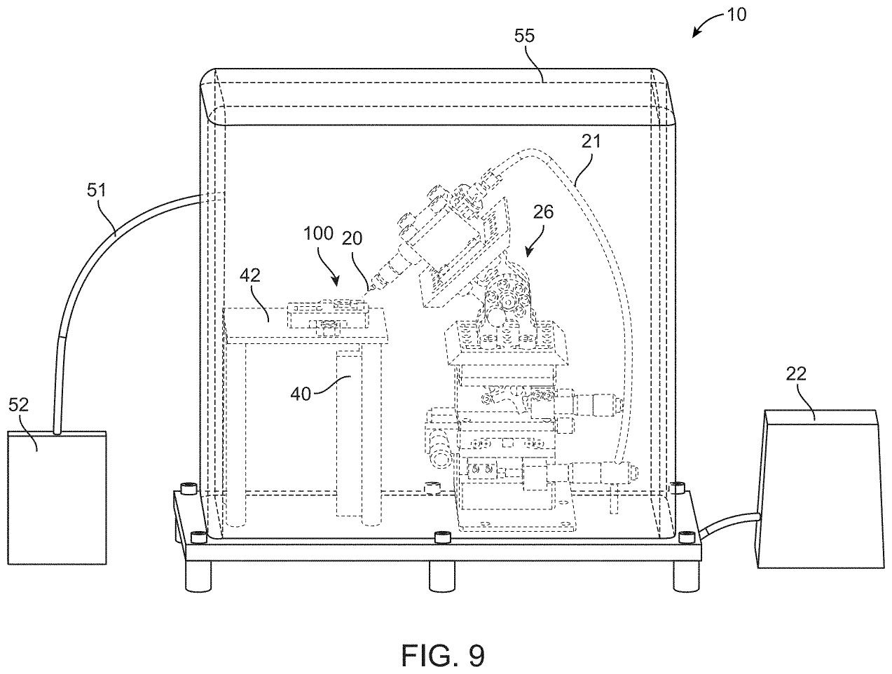

FIGS. 6A-6B, 7A-7D, 9, and 10A show implementations of the filling apparatus 10 in which the entire lens device 100, rather than just the internal chamber 155, is placed under vacuum conditions. The venting system 14 can include a vacuum pump 52 in fluid communication with a vacuum chamber 55 via tubing 51. The vacuum chamber 55 can be sized to contain at least the lens device 100 being held within the fixture 30 of the holding system 16. The vacuum pump 52 can create a vacuum within the vacuum chamber 55 so that the environment inside and outside the lens device 100, specifically inside and outside the dynamic membrane of the lens device 100, is the same. The dynamic membrane 143 in this scenario remains at neutral tension as air is removed from the chamber 155 and prior to filling. In other words, applying vacuum on both sides of the dynamic membrane 143 removes air from the chamber 155 as well as prevents deformation of the membrane 143 during manufacturing, except for the deformation achieved by filling with liquid to achieve a desired curvature or power. FIGS. 6A-6B, FIG. 7A, and FIG. 10A show a small vacuum chamber 55 sized to hold the lens device 100 positioned within the lens fixture 30. FIG. 9 shows a larger vacuum chamber 55 sized to hold the lens device 100 positioned within the lens fixture 30 plus one or more other components of the filling apparatus 10.

The vacuum chamber 55 can include a lid 201 sized and shaped to mate with a base 202 (see FIG. 10A). The lid 201 can be securely affixed to the base 202 using a clamp 200 or other mechanism. The clamp 200 ensures the lid 201 seals with the base 202 thereby allowing a vacuum to build in the chamber 55. The lid 201 of the vacuum chamber 55 can be positioned on the base 202 with the aid of a slider 203 or other mechanism that together with the clamp 200 allows a quick and easy opening and closing process for the vacuum chamber 55.

Again with respect to FIGS. 6A-6B, at least one wall 53 or a portion of the wall 53 of the vacuum chamber 55 can be made of a penetrable elastomeric material. The filling needle 20 of the dispensing system 12 can penetrate the wall 53 of the vacuum chamber 55 to access the injection zone 104 of the lens device 100 positioned inside the vacuum chamber 55. The angle of the filling needle 20 relative to the horizontal plane of the lens device is shown in FIG. 6B to be at an about 45 degree angle. It should be appreciated that the angle of filling needle 20 insertion can also be approximately parallel to the horizontal plane of the lens device (e.g., about 180 degrees) as discussed elsewhere herein. The vacuum pump 52 can maintain the desired negative pressure within the vacuum chamber 55 even when the filling needle 20 is inserted through the wall 53. As best shown in FIG. 6B, the venting system 14 can incorporate a vent needle 50 inserted through a region of the injection zone 104 to act as a ventilation port and provide a conduit for pressure equalization between inside the lens device 100 and the vacuum chamber 55. The vent needle 50 inserted into the venting port of the lens device 100 is not connected to the vacuum pump 52. Rather, the vent needle 50 connects the internal chamber 155 of the lens device 100 to the external surroundings so the air will be pumped from both simultaneously. A valve between the filling needle 20 and the vent needle 50 is incorporated to prevent oil entering the lens device through the filling needle 20 from exiting out the vent needle 50. Alternatively, a needle valve as described below can be incorporated for controlling oil entering the chamber through the filling needle 20. In use, the lens device 100 positioned within a lens fixture 30 can be positioned within the vacuum chamber 55. The lens device 100 can be penetrated with the vent needle 50 to provide the ventilation port. The vent needle 50 can be inserted using the rotation method described elsewhere herein to expose the distal opening of the vent needle 50 to the internal chamber 155 of the lens device 100. The vent needle 50 can be inserted along a path that is parallel the horizontal plane (i.e., 180 degrees) so as to avoid any manipulation of the needle, such as rotation after penetration of the chamber. The penetration zone for the vent needle is shown in FIG. 6B on a posterior side near a perimeter of the device. The penetration zone can also be located on a lateral side of the device as shown in FIG. 7B. The proximal end of the vent needle 50 can also have an opening that can be exposed to the environment of the vacuum chamber 55 providing a conduit for pressure equalization between the vacuum chamber 55 and the internal chamber 155 of the lens device 100. The filling needle 20 can be inserted into the lens device 100 as described elsewhere herein using the rotation method to ensure the distal opening 29 is fully within the internal chamber 155. The filling needle 20 can be inserted along a path that is parallel the horizontal plane (i.e., 180 degrees) so as to avoid any manipulation of the needle, such as rotation after penetration of the chamber. The injection zone is shown in FIG. 6B on a posterior side near a perimeter of the device. The injection zone can also be located on a lateral side of the device as shown in FIG. 7B. The vacuum pump 52 can be turned on to draw the air out of the vacuum chamber 55 and from the lens device 100 through the vent needle 50. The vacuum pump 52 can be turned off and the fluid dispenser 22 turned on to inject an amount of liquid from the dispensing system 12 into the internal chamber 155 of the lens device 100 to achieve a desired curvature or power. In other implementations, no vent needle 50 is used and the air from inside the lens device 100 escapes due to porosity of the silicone material of the device.

The lens device 100 can be placed within a first holder fixture for oil filling and then transferred to a second holder fixture for power evaluation. The lens device 100 can be transferred between the first and second holder fixtures depending on whether oil is being added to the lens device or the power is being evaluated. It is preferred to avoid transferring the lens device 100 between different holder fixtures. Thus, the lens device 100 can be placed in a single holder fixture and oil added to the lens device 100. The lens device 100 in the holder fixture can then be transferred to an optical bend for power measurements while remaining in the same holder fixture. If the power is not sufficient, the lens device 100 in the same holder fixture can be transferred back to the oil filling system and the amount of oil adjusted.

Still further, the lens device 100 need not be transferred between holder fixtures or the between the oil filling and power measurement benches. In this configuration, the curvature or power can be measured in real-time during filling with the measurement system 18. The probe 40 of the measurement system 18 can access the lens zone 102 of the lens device 100 within the vacuum chamber 55. The vacuum chamber 55 can allow for visibility of the injection port from the side and from above using a camera to monitor the curvature or power of the lens device during filling. The probe 40 can be positioned inside or outside the vacuum chamber 55 in order to make a reading. In some implementations, the probe 40 is outside the chamber 55 and the lens holder and the stage 42 each have an aperture through which the probe 40 can extend to make a reading of the curvature or power. The probe 40 can also read through a glass covering of the aperture(s) such as a cuvette 36 as shown in FIG. 10C.

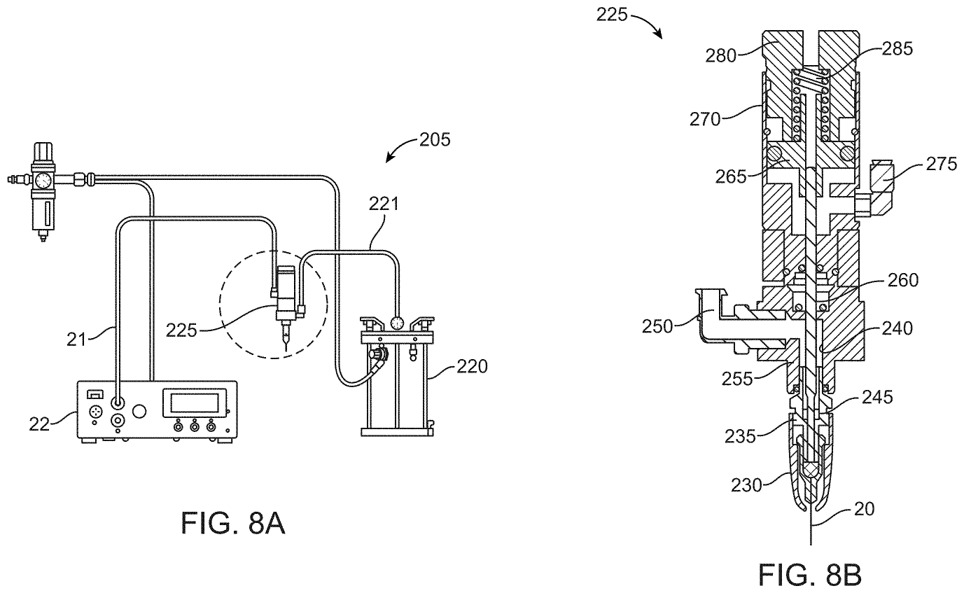

As mentioned above, the vent needle 50 can incorporate a valve to prevent oil entering the chamber from exiting out the vent needle 50. The oil entering the chamber may be measured to prevent it from exiting. Also, the oil is relatively viscous and unlikely to flow through a small-sized needle (e.g., 32 G or 0.1 mm diameter). In other implementations, the filling apparatus 10 can include a dispensing system 12 having a filling needle 20 that is coupled to a needle valve system 205 (see FIGS. 7A-7D and also FIGS. 8A-8B). The needle valve system 205 can include an adjustable stroke needle valve 225 that is pneumatically triggered single-acting valve that can be used with an internal spring close (Techcon no. TS5440). As shown in FIG. 7A, the needle valve system 205 is connected to a dispenser 22, such as an air dispenser that provides a pulse of air at the desired time and pressure. The needle valve system 205 is also connected to a syringe 215 filled with the oil to be dispensed into the lens device through the filling needle 20. The oil syringe 215 is connected to a pressure tank 220 that applies a constant pressure (e.g., 4-5 psi).

FIG. 8A is a schematic of the connections between the needle valve 225 and the rest of the needle valve system 205. The needle valve 225 is coupled to pulsed air via the dispenser 22 and to the oil via the pressure tank 220 and oil line 221. FIG. 8B is a cross-sectional schematic view of the needle valve 225. The distal end of the needle valve 225 couples to the filling needle 20 that projects distally beyond a luer cap 230. The luer cap 230 couples to a luer taper 235. A flow channel 240 is defined through a needle valve seat 245 to control flow through the filling needle 20. A fluid inlet 250 is coupled to the body 255 of the needle valve 225 and is configured to be in fluid communication with the flow channel 240. The needle 260 is coupled to a piston 265 at a proximal end of the needle 260. The piston 265 and needle 260 are movable within an air cylinder 270 upon receipt of air via the air inlet 275. A flow control adjustment 280 is positioned in a proximal end of the air cylinder 270 to adjust the amount of fluid being dispensed through the filling needle 20. The piston 265 can be returned via a spring 285. The spring immediately returns the needle 260 in the closed position. The flow control adjustment 280 controls the length of the spring and the speed at which the return occurs. If the spring length is set at zero, for example, the needle valve does not open.

When the needle valve 225 is closed oil is prevented from flowing through the filling needle 20, which allows for building the vacuum within the vacuum chamber 55 without uncontrolled oil flow. The needle 260 of the needle valve 225 closes flow through the flow channel 240 by resting in a distal position against the needle seat 245. The needle valve 225 is connected to the dispenser 22 via line 21. A user may actuate the needle valve 225, such as by pressing a foot switch or other actuator. The air flows through the air inlet 275 into the air cylinder 270 and retracts the needle 260 away from the needle seat 245 urging the piston 265 proximally thereby compressing the spring 285. This opens the flow channel 240 to allow liquid from the fluid inlet 250 to flow around the needle 260 near the seat 245 and out the filling needle 20. Input air pressure is relieved allowing the spring 285 to urge the piston 265 distally seating the needle 260 against the needle seat 245 shutting off flow through the flow channel 240.

FIGS. 7B and 7C show the filling needle 20 penetrating the lens device positioned within the vacuum chamber 55 and held within a holding system 16 to maintain the position of the lens device 100 relative to the dispensing system 12 as discussed elsewhere herein. The position of the filling needle 20 can be moved relative to the lens device 100 as described elsewhere herein, such as by a motorized precision gantry system to achieve the desired spatial orientation of the filling needle 20 relative to the lens device 100. The vent needle 50 in fluid communication with the vacuum pump 52 configured to draw air out of the lens device 100 may also be positioned using a precision gantry system.

FIG. 9 illustrates an implementation of the vacuum chamber 55 sized larger so as to contain one or more other components of the filling apparatus 10. Here, the vacuum chamber 55 is sized to contain the holding system 16, the measurement system 18, and at least some of the dispensing system 12 including the filling needle 20 and its gantry 26. The vacuum pump 52 and the dispenser 22 can be located physically outside the vacuum chamber 55. The vacuum pump 52 can be placed in fluid communication with the environment of the vacuum chamber 55 through tubing 51. Similarly, the dispenser 22 can be placed in fluid communication with the filling needle 20 via tubing 21.

The filling and/or withdrawal of liquid from the internal chamber can be under the control of an electronics module 500 as described above. The apparatus 10 can be programmed for control of the filling using the dispensing system and adjusted during filling as advised by the measurement system 18.

The lens device 100 can be filled with liquid optical material at the time of manufacturing and prior to insertion into a patient. The lens device 100 can be filled to contain a specific amount of the liquid in the chamber and evaluated, such as in real-time during filling. The amount of liquid within the chamber can be adjusted during the evaluation. After an initial percentage of the total liquid is dispensed within the chamber, the measurement system can scan the dynamic membrane (or optical surface) of the lens device, calculate the radius of curvature, and then calculate how much volume of liquid is needed to reach the final dispense level to achieve the desired radius of curvature. The dispenser can be controlled by the measurement system to fill the exact volume needed to achieve the desired radius of curvature. The dynamic membrane (or any optical surfaces that are movable) of the lens device can be maintained in a pressure neutral environment such that the movable components of the lens device do not deform significantly as the air is removed. The vacuum chamber can create a similar pressure inside and outside of the lens device as the air is removed. Once the internal chamber 155 of the lens device 100 is filled with the liquid, the lens device 100 can be folded or otherwise prepared for injection into the eye using any of a variety of insertion devices known in the art.

Again with respect to FIGS. 2A-2B, an exemplary lens device 100 is showing having a flexible anterior optic 145. The lens device of FIGS. 2A-2B is illustrated as one of any of a variety of lens devices that can be filled using the apparatus 10 described herein as discussed elsewhere herein.

The anterior optic 145 can be a flexible optic formed of an optically clear, low modulus polymeric material, such as silicone, polyurethane, or flexible acrylic. The anterior optic 145 can include a static anterior optical portion 144 surrounding a central, dynamic membrane 143 configured to outwardly bow as discussed elsewhere herein. The dynamic membrane 143 can be positioned relative to the lens body 105 such that the optical axis A of the lens extends through the dynamic membrane 143. The anterior optic 145 can have a variable thickness. For example, the dynamic membrane 143 can have a reduced thickness compared to the static anterior optical portion 144. The thinner cross-sectional thickness of the dynamic membrane 143 compared to the cross-sectional thickness of the static anterior optical portion 144 can render it relatively more prone to give way upon application of a force on its inner surface. For example, upon an increased force applied against inner surfaces of the anterior optic 145 during deformation of the fluid chamber 155, the dynamic membrane 143 can bow outward along and coaxial to the optical axis A of the lens 100 while the static anterior optical portion 144 maintains its shape. The dynamic membrane 143 can be configured to give way due to pressure applied by the liquid optical material within the fluid chamber 155 onto the internal surface of the anterior optic 145 causing an outward bowing of the outer face (e.g., anterior face). Outer static anterior optical portion 144 of the anterior optic 145 can have a thickness greater than the inner dynamic membrane 143 of the optic 145 and can be more resistant to reshaping under such internal pressure applied by the liquid optical material in the fluid chamber 155. The outer static anterior optical portion 144 of the anterior optic 145 can provide distance vision correction even when the inner dynamic membrane 143 is reshaped for near vision. The dynamic membrane 143 can have a substantially constant thickness such that it is a planar element. Preferably, the dynamic membrane 143 can have a variable thickness between its outermost edge and central region as discussed in more detail above and as shown in FIG. 2B. The dynamic membrane 143 can have a linear gradient thickness, curved gradient thickness, 2, 3 or more thicknesses with a step including radiused or right angles.

The dynamic membrane 143 can also include multiple materials, for example, materials configured to flex near a center of the dynamic membrane 143 and other materials configured to reinforce the optic zone and limit distortion. Thus, the dynamic membrane 143 of the anterior optic 145 can be formed of a material that is relatively more susceptible to outward bowing than the material of outer static anterior optical portion 144. The various regions of the optic 145 can be injection or compression molded to provide a relatively seamless and uninterrupted outer face. The material of the regions can be generally consistent, though the dynamic membrane 143 can have different stiffness or elasticity that causes it to bow outward farther than the static anterior optical portion 144.

The anterior optic 145 can be configured to have varied multifocal capabilities to provide the wearer of the lenses described herein with enhanced vision over a wider range of distances, for example, as described in U.S. Publication No. 2009/0234449, which is incorporated by reference herein in its entirety. The “optic zone” as used herein generally refers to a region of the lens body 105 that surrounds the optical axis A of the lens and is optically clear for vision. The “accommodating zone” as used herein generally refers to a region of the lens body 105 capable of undergoing shape change for focusing (e.g., the dynamic membrane 143). The optic zone is configured to have a corrective power although the entire optic zone may not have the same corrective power. For example, the dynamic membrane 143 and the static anterior optical portion 144 of the anterior optic may each be positioned within the optic zone. The dynamic membrane 143 may have corrective power whereas the static anterior optical portion 144 may not have corrective power. Or, for example, the diameter defined by the dynamic membrane 143 may have an optical power and the static anterior optical portion 144 may have a power that is greater or lesser than that of the dynamic membrane 143. The dynamic membrane 143 can be equal to or smaller than the overall optical zone can create a multi-focal lens. The accommodating zone of the lens body 105 can be equal to or smaller than the overall optic zone.

A shape deformation membrane 140 can extend along an arc length of the equator region of the lens body 105. The arc length can be sufficient, either individually or in combination with other shape deformation membranes 140, to cause a reactive shape change in the dynamic membrane 143 upon inward (or outward) movement of the deformation membrane 140. Movement of the shape deformation membrane 140 in a generally inward direction towards the optical axis A of the lens 100 during accommodation can cause outward flexure or bowing of the dynamic membrane 143 without affecting the overall optic zone diameter in any axis.

The shape deformation membrane 140 can have a flexibility such that it is moveable and can undergo displacement relative to the lens body 105, the static element 150, and the anterior optic 145. For example, the shape deformation membrane 140 can be more flexible than adjacent regions of the lens body 105 such that it is selectively moveable relative to the lens body 105 and the static anterior optical portion 144 of the anterior optic 145. The shape deformation membrane 140 can have a resting position. The resting position of the shape deformation membrane 140 can vary. In aspects, the resting position is when the shape deformation membrane 140 is positioned generally perpendicular to a plane parallel to the anterior optic 145 such that it has a cross-sectional profile that is vertically oriented, parallel to the optical axis A. The resting position of the shape deformation membrane 140 can also be angled relative to the optical axis A of the lens body 105. The shape and relative arrangement of the one or more side deformation membranes 140 provides the lens with a low force, low movement, high accommodative function.

The movement of the shape deformation membrane 140 can be a compression, collapse, indentation, stretch, deformation, deflection, displacement, hinging or other type of movement such that it moves in a first direction (such as generally toward an optical axis A of the lens body 105) upon application of a force on the shape deformation membrane 140.

The shape deformation membrane 140 lies adjacent or is coupled to or molded integral with a respective force translation arm 115. The one or more force translation arms 115 are configured to harness movements of one or more of the ciliary structures such that they are bi-directionally movable relative to the lens body 105 to effect accommodative shape change of the lens body 105. For example, and without limiting this disclosure to any particular theory or mode of operation, the ciliary muscle is a substantially annular structure or sphincter. In natural circumstances, when the eye is viewing an object at a far distance, the ciliary muscle within the ciliary body relaxes and the inside diameter of the ciliary muscle gets larger. The ciliary processes pull on the zonules, which in turn pull on the lens capsule around its equator. This causes a natural lens to flatten or to become less convex, which is called disaccommodation. During accommodation, the ciliary muscle contracts and the inside diameter of the ring formed by the (ciliary ring diameter, CRD) ciliary muscle gets smaller. The ciliary processes release the tension on the zonules such that a natural lens will spring back into its natural, more convex shape and the eye can focus at near distances. This inward/anterior movement of the ciliary muscle (or one or more ciliary structures) can be harnessed by the force translation arms 115 to cause a shape change in the lens body 105.

In aspects, as the force translation arm 115 is moved inwardly toward the optical axis A of the lens 100 due to ciliary muscle contraction, the force translation arm 115 abuts an outer surface of the shape deformation membrane 140 and applies a force against the outer surface. Thus, the contact between the shape deformation membrane 140 and the force translation arm 115 can be reversible contact such that upon ciliary muscle contraction the force translation arm 115 is urged against the outer surface abutting the membrane 140 and urging it inwardly. Upon ciliary muscle relaxation, the shape deformation membrane 140 returns to its resting position and the force translation arm 115 returns to its resting position. The elastomeric nature of the movable components (i.e., the dynamic membrane and/or the shape deformation membranes) can cause a return of the force translation arms 115 to their resting position. In aspects, the shape deformation membrane 140 is coupled to or integral with its respective force translation arm 115. As with other aspects, upon ciliary muscle contraction the force translation arm 115 and shape deformation membrane 140 move in concert from a resting position to a generally inwardly-displaced position causing shape change of the dynamic membrane 143. Displacement of the force translation arm 115 and associated shape deformation membrane 140 applies a compressive force on the fluid chamber and in turn deforms the chamber causing the dynamic membrane 143 to bulge outward.

The inward motion of the force translation arm 115 and associated shape deformation membrane 140 can be coaxial to an axis that is substantially orthogonal or perpendicular to the optical axis A. Meaning, the angle between the axis of motion and the optical axis can be 90 degrees plus or minus about 1 degree, 2 degrees, 3 degrees, 4 degrees, up to about 5 degrees. It should be appreciated that a compressive force applied to the force translation arms 115, such as by a ciliary structure may result in radially inward motion that is not perfectly orthogonal to the optical axis A and that some degree greater than or less than 90 degrees is considered herein. The angle between the axis of motion of the deformation membrane 140 and the optical axis A can also be substantially non-orthogonal or non-perpendicular. For example, the deformation membrane 140 can be compressed along an axis that is non-orthogonal to the optical axis A.

The number and arc length of each deformation membrane 140 can vary and can depend on the overall diameter and thickness of the device, the internal volume, refractive index of the material, etc. Generally, the lens body has sufficient rigidity and bulk to the lens such that it can be handled and manipulated during implantation while the deformation membrane(s) 140 are sufficiently flexible to allow the force translation arms to change the shape of the fluid chamber 155. Depending on the overall diameter and thickness of the lens 100, the arc length of the shape deformation membrane 140 can be at least about 2 mm to about 8 mm. In aspects, the lens has a single shape deformation membrane 140 with an arc length of between about 2 mm to about 8 mm. The single shape deformation membrane 140 can be designed to move between about 10 μm and about 100 μm upon application of forces as low as about 0.1 grams of force (gf) to achieve at least a 1D, or 1.5D, or 2D, or 2.5D, or 3D change in the dynamic membrane 143. In aspects, the IOL can have two, opposing shape deformation membranes 140 each having an arc length that is between about 3 mm and about 5 mm. The shape deformation membranes 140 can be designed to move between about 25 μm and about 100 μm each upon application of about 0.25 g force to 1.0 g force achieve at least a 1D change in the dynamic membrane 143.

The shape deformation membranes 140 can move or collapse relative to the rest of the lens body upon application of a degree of compressive force. Generally, the IOL is designed such that very low forces (including the application of compressive force towards the optical axis A as well as the release of the compressive force) are sufficient to cause micron movements to cause sufficient diopter changes and with reliable optics. The compressive force applied to achieve outward movement of the dynamic membrane 143 of the lens body 105 to effect accommodation can be as low as about 0.1 grams of force (gf). In aspects, the compressive force applied can be between about 0.1 gf to about 5.0 gf or between about 0.25 gf to about 1.0 gf or between about 1.0 gf to about 1.5 gf. The movements of the deformable regions of the lens body 105 (e.g., shape deformation membrane 140) relative to the central portion of the lens body 105 (e.g., dynamic membrane 143) in response to the compressive forces applied to achieve accommodation can be as small as about 50 μm. The movements of the shape deformation membrane 140 of the lens body relative to the dynamic membrane 143 in response to the compressive forces applied can be between about 50 μm to about 500 μm, between about 50 μm to about 100 μm, between about 50 μm to about 150 μm, or between about 100 μm to about 150 μm. The ranges of compressive forces applied (e.g., about 0.1 gf to about 1 gf) that result in these ranges of movement in the shape deformation membrane 140 (e.g., 50 μm-100 μm) can provide the devices described herein with an accommodating capability that is within a dynamic range of greater than at least ±1D and preferably about ±3 diopters (D). In aspects, the power is between ±4D and ±6D for about 100-150 μm movement. The devices described herein can have an accommodating range that is at least ±1D for about 100 μm movement of the shape deformation membrane 140 and about a compressive force of at least 0.25 gf applied to the shape deformation membrane 140 in a substantially inward direction towards the optical axis A. In aspects, the devices can have an accommodating range that is at least ±1D for about 50 μm movement and at least about 1.0 gf. In aspects, the devices can have an accommodating range that is at least ±3D for about 100 μm movement and at least about 1.0 gf. In aspects, the devices can have an accommodating range that is at least ±3D for about 50 μm movement and at least about 0.1 gf.

The micron movements described herein can be asymmetrical micron movements (e.g., from one side of the device) or can be symmetrical micron movements from opposing sides of the device or evenly distributed around the device relative to the optical axis. Whether the micron movements are asymmetric or symmetrical, the outward bowing of the dynamic membrane 143 achieved can be substantially spherical. The micron movements described herein also can be a total collective movement of the shape deformation membranes 140. As such, if the lens 100 includes a single shape deformation membrane 140, that single membrane is capable of desired micron movement (e.g., 50 μm-100 μm) to achieve desired dioptric change (e.g., at least 1D to about 3D change). If the lens 100 includes two shape deformation membranes 140, the membranes together are capable of the achieving between 50 μm-100 μm movement to achieve the at least 1D dioptric change. The dioptric change achieved by the devices described herein can be at least about 1D up to approximately 5D or 6D change. In aspects, the dioptric change can be between 7D and 10D, for example, for patients having macular degeneration.

The static element 150 and the anterior optic 145 can be located opposite one another along the optical axis A of the lens 100. The static element 150 can be positioned outside the lens body 105 such that the flat surface forms the inner surface facing the fluid chamber 155 of the lens body 105 and the curved surface is in contact with the fluid of the eye. Alternatively, the static element 150 can be positioned inside the lens body 105 such that the flat surface is in contact with the fluid of the eye and the curved surface forms the inner surface facing the fluid chamber 155 of the lens body 105.

The static element 150 can be optically clear and provide support function without affecting the optics of the lens 100. As such, the static element 150 can have zero power and can form a posterior support to the lens body 105. The static element 150 can be formed of silicone, urethane, acrylic material, a low modulus elastomer, or combinations thereof. The static element 150 can be or include a static optic to correct to emmetropic state, or can be of an appropriate power for an aphakic patient (usually ±10D to ±30D). Thus, the static element 150 can have no optical power up to about ±30D. If the lens 100 is being used in conjunction with a separate capsular lens (e.g., as a “piggyback” lens), the power can be in the range of about −5D to about +5D to correct for residual refractive or other optical aberrations in the optical system of the eye. The static element 150 can be plano-convex, convex-plano, convex-convex, concave-convex or any other combination. The static element 150 (or the lens positioned posteriorly) can be a toric lens, spherical lens, aspheric lens, diffractive lens or any combination of both, for example, in order to reduce or compensate for any aberrations associated to the flexible lens. The relative refractive indices of the static element 150 and the fluid surrounding it (whether that is the fluid of the eye or liquid optical material within the fluid chamber 155) will determine the power of the static element 150 for any given shape.

The lens 100 can include any of a variety of combinations of reinforcements and/or supports to provide mechanical stability to the assembled lens 100. For example, the reinforcements may be in the peripheral regions of the anterior lens 145 and/or the static element 150. The reinforcements can be either optically clear or opaque. The reinforcing structures may be formed of a rigid polymer, including but not limited to silicone, polyurethane, PMMA, PVDF, PDMS, polyamide, polyimide, polypropylene, polycarbonate, etc., or combinations thereof. Other regions of the lens 100 can include one or more reinforcements or supports as well. In aspects, the one or more supports can be positioned external to the fluid chamber 155 such that the supports surround at least an outside portion of the lens body 105. For example, the external support can be a generally annular element extending around a perimeter of the lens body 105 and have a central opening through which at least the dynamic membrane 143 of the anterior optic 145 is aligned such that the dynamic membrane 143 is available for outward deformation.

The sealed, fixed volume fluid chamber 155 can be collectively formed by the inner-facing surfaces of the shape deformation membrane 140, the anterior optic 145, and the static element 150. The chamber 155 can be filled using the filling apparatus 10 described herein with a volume of a liquid optical material.

The liquid optical material contained within the fluid chamber 155 can be a non-compressible liquid optical material and the volume of the fluid chamber 155 can be substantially identical to the volume of liquid optical material. As such, the liquid optical material contained within the chamber 155 does not cause significant outward bowing of either the dynamic membrane 143 or the deformation membrane 140 in the resting state when no substantial outside forces are applied to the lens 100. In aspects, the fluid chamber 155 can be slightly overfilled with liquid optical material such that the dynamic membrane 143 has some outward bowing at rest. A small degree of resting outward bowing in the dynamic membrane 143 can reduce optical artifacts in the lens. The filling apparatus 10 described in detail above can inject a precise volume of liquid optical material into the chamber 155 of the lens 100 so that the resting bowing achieved by filling is suitable for desired optics. However, no matter how much resting outward bowing is built into in the dynamic membrane 143, the membrane 143 can still undergo additional outward bowing upon implantation in the eye and upon application of compressive forces on the shape deformation membrane 140 to provide accommodation.

The pressure inside the fluid chamber 155 can be substantially equal to the pressure outside the fluid chamber 155. Because the liquid optical material in the fluid chamber 155 is non-compressible its shape deforms along with the shape of the chamber 155. Deformation of the chamber 155 in one location (e.g., micrometer inward movements of the shape deformation membrane 140) causes the non-compressible liquid optical material contained within the fixed-volume fluid chamber 155 to press against the inner-facing surfaces forming the fluid chamber 155. A reactive deformation of the fluid chamber 155 occurs in a second location to create sufficient accommodating change. The dynamic membrane 143 of the anterior optic 145 is configured to bow outward upon application of a force (e.g., due to relative thickness and/or elasticity) compared to other parts of the anterior optic 145, such as the static anterior optical portion 144. Thus, inward movement of shape deformation membrane 140 urges the liquid optical material to deform along with the chamber 155 and press against the inner-facing surface of the anterior optic 145. This results in outward bowing and reshaping of the outer surface of the dynamic membrane 143 to cause the accommodative portion of the optic zone to become more convex increasing the power of the lens 100.

The liquid optical material contained within the fluid chamber 155 of the lens body 105 remains substantially within the optic zone during rest in both the unaccommodated, resting state and during accommodation. The liquid optical material remains within the lens body 105 and can contribute to the accommodative shape change of the dynamic membrane 143 by deforming in shape along with the deformation of the shape of the fluid chamber 155. It should be appreciated that this shape change of the dynamic membrane 143 can occur without actual flow of the liquid optical material within the fluid chamber 155, for example, from one part of the chamber to another. Rather, a force being applied on the shape deformation membrane 140 deforms the fluid chamber 155 in a first region that can cause a reactive deformation of the fluid chamber 155 in at least a second region. The fluid chamber 155 has a fixed volume and is deformable. The liquid optical material contained within the fluid chamber 155 changes shape along with and depending on the shape of the fluid chamber 155. Inward deformation of one or more portions of the chamber 155, for example, movement of the shape deformation membrane 140 near the static zone of the lens body 105, can cause a reactive outward deformation of another portion of the chamber 155, for example, outward bulging of the dynamic membrane 143 of the anterior optic 145, due to the non-compressible liquid optical material inside the fluid chamber 155 pressing against its inner surface. The liquid optical material need not flow between separate chambers of the IOL, but rather the liquid optical material can change shape along with the changing shape of the fluid chamber 155 to cause the accommodative portion of the optic zone of the anterior optic 145 to bow outward and increase the power of the IOL 100. As described elsewhere herein, very small movements of the force translation arms 115 (or single force translation arm 115 in the case of an asymmetric mechanism) result in immediate, small movements in the shape deformation membrane 140 to change the shape of the dynamic membrane 143 and sufficient dioptric change. Whether these very small movements are symmetrical due to at least a pair of opposing force translation arms 115 or asymmetrical due to a single force translation arm 115, the outward bowing of the dynamic membrane 143 that is achieved is spherical and symmetrical. The shape deformation membrane 140 is sensitive to small forces imparted on the lens body 105. This is useful in providing accommodative changes upon ciliary muscle movements. However, this can cause power changes with undesirable optical consequences if the liquid optical material migrates away from the fluid chamber 155, for example, into the surrounding solid optical components. It is preferred that the liquid optical material be chemically dissimilar enough to prevent miscibility with the solid optical components it comes into contact with. For example, if the liquid optical material is a silicone oil and the sealed chamber 155 is defined by solid optical components formed of a chemically similar silicone elastomer like polydimethylsiloxane (PDMS), the silicone oil and silicone elastomer are miscible. The oil tends to enter into the silicone elastomer causing an unintended optical power change in the lens. The surface curvatures of the lens body would decrease (less convex or more concave) thereby reducing the power of the lens and providing insufficient optical power to the patient. This also reduces the ability of the lens to undergo sufficient shape change when necessary at the time of accommodation. Even minor changes of the internal pressure can result in substantial undesirable changes to the optical power of the lens.

The lens 100 can also include a stabilization system 120. The stabilization system 120 can be configured to maintain alignment of the optics of the device and resist movement of the device once the device is implanted and undergoing shape changes. Unlike the force translation arms 115, the stabilization system 120 does not cause accommodation of the lens 100. And because the force translation arms 115 are independent from the stabilization system 120 and are not necessary to fix, center, stabilize, and/or hold the lens 100 in position within the eye, the lenses 100 described herein can incorporate a single, asymmetric force translation arm 115 sufficient to provide the dioptric change of the dynamic membrane. The stabilization system 120 can be coupled to a static zone of the device 100, for example, bonded, coupled, or molded as part of the lens body 105 or to an exterior support, if present. The stabilization system 120 can be coupled to a posterior region of the device 100 such that it can provide stabilization and engagement with a portion of the capsular bag, such as with the anterior capsule. The stabilization systems can be formed from silicone elastomer, polyurethane, PMMA, PVDF, PDMS, polyamide, polyimide, polypropylene, polycarbonate, or flexible acrylic materials that are hydrophobic or hydrophilic or any combination of those materials. The stabilization system may have a softer body that is reinforced with more rigid structures in order to provide its stabilizing function while maintaining flexibility for insertion and manipulation. One or more portions of the stabilization system 120 described herein can incorporate biting elements to improve fixation within the eye. In aspects, the stabilization system 120 includes haptics and the biting elements can be positioned near their terminal ends to improve fixation of the haptic within the eye. The stabilization haptics can be any of a variety of haptic designs or combination of haptic designs including, but not limited to open-loop, closed-loop, plate-style, plate loop, monoblock-plate style, j-loop, c-loop, modified J-loop, multi-piece, single-piece, angulated, planar, offset, etc. Haptics considered herein can include the Rayner designed haptics (Rayner Intraocular Lenses Ltd, East Sussex, UK), NuLens designed haptics (NuLens Ltd., Israel), Staar lens designs (Staar Surgical, Monrovia, Calif.), and others. In aspects, the stabilization system 120 whether including one or more haptics or a 360 degree wing can be formed of a biocompatible polymer, such as silicone, polyurethane, PMMA, PVDF, PDMS, polyamide, polyimide, polypropylene, polycarbonate, PEEK, etc. or a combination of such materials. The stabilization system 120 can be formed of a material or configured to be foldable. In aspects, the stabilization system 120 is formed of a shape memory material.

The various components and features of the lenses described herein can be incorporated in any of a variety of combinations. As such, description of a particular feature shown with respect to a particular drawing is not intended to be limiting in that the feature can be incorporated into another implementation of a lens described herein. For example, the lenses described herein can include a stabilization system that incorporates one or more features of the stabilization systems described herein. Further, the lens having the stabilization system features can be combined with any of a variety of features described with respect to the force translation arm 115 or the shape deformation membrane 140, for example.

Suitable materials or combinations of materials for the preparation of the various solid optical components of the devices disclosed herein are provided throughout. It should be appreciated that other suitable materials are considered. U.S. Patent Publication Nos. 2009/0234449, 2009/0292355 and 2012/0253459, which are each incorporated by reference herein in their entirety, provide further examples of other materials suitable for forming certain components for the devices described herein. One or more solid optical components of the lens body 105 can be integral with one another in that they are formed of the same material. Alternatively, one or more of the solid optical components of the lens body 105 can be coupled together by techniques known in the art. In aspects, the liquid optical material contained within the fluid chamber 155 can be a fluorosilicone oil and the solid optical components forming the fluid chamber 155 are formed of a silicone elastomer. In aspects, the liquid optical material contained within the fluid chamber 155 is a silicone oil and the solid optical components forming the fluid chamber 155 are formed of a fluorosilicone elastomer. In aspects, the liquid optical material contained within the fluid chamber 155 is an aromatic or phenyl-substituted oil, such as phenylsilicone oil and the solid optical components forming the fluid chamber 155 are formed of a halogenated silicone elastomer, such as fluorosilicone elastomer. The combinations of materials are chosen to optimize stability of the lens, prevent swelling and maintaining optimum refractive index.

In aspects, the force translation arms 115 can be a rigid polymer formed of silicone, polyurethane, PMMA, PVDF, PDMS, polyamide, polyimide, polypropylene, polycarbonate, etc., or combinations thereof. In some implementations, the force translation arms 115 can be an element reinforced with PMMA. In aspects, the lens is formed of all silicone materials including the posterior static element 150 and the force translation arms 115. The stabilization system 120 can be formed of a more rigid silicone or can be formed of or incorporate polyimide.

The lenses described herein can provide focusing power across the full accommodative range from distance to near by mechanically and functionally interacting with eye tissues typically used by a natural lens, such as the ciliary body, ciliary processes, and the zonules, to effect accommodation and disaccommodation. The devices described herein can include an accommodative mechanism including one or more force translation arms configured to be positioned in the eye such that they harness movements of one or more ciliary structures and translate the movements into functional forces to drive shape change of the lens body for accommodation and disaccommodation in a manner independent of capsular bag movements. The lenses described herein can achieve an optical power change in the range of 1 diopter (1D) to 3D up to about 5D or 6D. The forces generated by these tissues are functionally translated to the devices described herein causing a power change to more effectively accommodate. The lenses described herein can further include a stabilization system separate from the accommodative mechanism that is configured to be positioned, for example, within the capsular bag. The devices described herein obviate known issues that tend to occur due to capsular fibrosis described above. It should be appreciated that the devices described herein can be configured to harness movements of one or combinations of ciliary structures including, but not limited to, the ciliary muscle, the ciliary body, ciliary processes, and zonules. For the sake of brevity, the term “ciliary structure” may be used herein to refer to any of the one or more ciliary structures for which movements can be harnessed by the force translation arms to effect accommodation of the lens body.

The devices described herein can be implanted in the eye to replace a diseased, natural lens. The devices can be implanted as a supplement of a natural lens (phakic patient) or an intraocular lens previously implanted within a patient's capsular bag (pseudophakic patient). The lenses described herein can be used in combination with intraocular lenses described in US 2009/0234449, US 2009/0292355, US 2012/0253459, U.S. Pat. No. 10,285,805, US 2019/0269500, which are each incorporated by reference herein in their entirety. As such, the lenses described herein can be used independently or as so-called “piggyback” lenses. Piggyback lenses can be used to correct residual refractive errors in phakic or pseudophakic eyes. The primary lens used to replace the natural lens is generally thicker and usually has a power that can be in the range of ±10D to ±25D. The thicker, larger power lenses generally do not accommodate. In contrast, the supplemental lens need not provide significant optical power to the system. The supplemental lens can be relatively thin compared to the primary lens and can undergo more accommodation. Shape change and movement of the thinner lens is generally more easily accomplished relative to a thick primary lens. The lenses described herein can be used independently and need not be used in combination as piggyback lenses with the natural lens or an implanted lens. One or more components of the lenses described herein can be configured to be positioned in the sulcus, against the ciliary processes, within the capsular bag or a combination thereof.

The devices and systems described herein can incorporate any of a variety of features. Elements or features of one implementation of a device and system described herein can be incorporated alternatively or in combination with elements or features of another implementation of a device and system described herein as well as the various implants and features described in US 2009/0234449, US 2009/0292355, US 2012/0253459, U.S. Pat. No. 10,258,805, and US 2019/0269500, which are each incorporated by reference herein in their entireties. For the sake of brevity, explicit descriptions of each of those combinations may be omitted although the various combinations are to be considered herein. The various devices can be implanted, positioned and adjusted etc. according to a variety of different methods and using a variety of different devices and systems. The various devices can be adjusted before, during as well as any time after implantation. Provided are some representative descriptions of how the various devices may be implanted and positioned, however, for the sake of brevity explicit descriptions of each method with respect to each implant or system may be omitted.

In aspects, description is made with reference to the figures. However, certain aspects may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detain in order to not unnecessarily obscure the description. Reference throughout this specification to “one embodiment,” “an embodiment,” “an aspect,” “one aspect,” “one implementation, “an implementation,” or the like, means that a particular feature, structure, configuration, or characteristic described is included in at least one embodiment, aspect, or implementation. Thus, the appearance of the phrase “one embodiment,” “an embodiment,” “one aspect,” “an aspect,” “one implementation, “an implementation,” or the like, in various placed throughout this specification are not necessarily referring to the same embodiment, aspect, or implementation. Furthermore, the particular features, structures, configurations, or characteristics may be combined in any suitable manner in one or more implementations.

The use of relative terms throughout the description may denote a relative position or direction or orientation and is not intended to be limiting. For example, “distal” may indicate a first direction away from a reference point. Similarly, “proximal” may indicate a location in a second direction opposite to the first direction. Use of the terms “front,” “side,” and “back” as well as “anterior,” “posterior,” “caudal,” “cephalad” and the like or used to establish relative frames of reference, and are not intended to limit the use or orientation of any of the devices described herein in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

Aspects of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device. For example, a software program can be incorporated into the apparatus that can measure power or radius of curvature of a particular portion of the lens device. The software can correlate the power or radius of curvature to the fill level and/or volume of liquid dispensed into the internal chamber and determine a total volume needed to achieve a desired target power or radius of curvature for the patient during filling in real-time so that the apparatus can automatically adjust the dispensed amount. Such a software program can be used to measure fill volume, radius of curvature, power of a lens in real-time. The software program can automatically control the dispense rate of liquid into the lens device based on the measurement data from the measurement system 18 that assess radius of curvature of the lens during filling or power of the lens or volume of fluid in the lens. The measurement system 18 can provide data that is used to control the flow of dispensed liquid into the lens device. Removing the air from the lens device while the optical element is maintained in a pressure-neutral environment prevents inadvertent and damaging stretching of the optical surfaces. Once the air is removed, the lens device can be filled with liquid while the measurement system monitors the changing geometry and power of the lens device. The apparatus provides for a lens device that is more precise in its refractive power and that has reduced optical aberrations.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples, embodiments, aspects, and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

1. A filling apparatus for filling a lens device with a volume of optical liquid, the filling apparatus comprising:
a dispensing system comprising a source of optical liquid, a dispenser, and a filling needle having a lumen in fluid communication with the source of optical liquid, the filling needle configured to penetrate an injection zone of the lens device for filling an internal chamber of the lens device with the volume of optical liquid;
a venting system comprising a vacuum pump and a vacuum chamber configured to be in fluid communication with the vacuum pump;
a measurement system configured to measure a lens zone of the lens device; and
a lens device holding system comprising a lens fixture for maintaining a position of the lens device relative to the filling needle and the measurement system.

2. The apparatus of claim 1, wherein the vacuum pump is configured to remove air from within the internal chamber during filling with the filling needle.

3. The apparatus of claim 1, wherein the vacuum pump is fluidly coupled to a proximal opening of a vent needle having a distal end configured to penetrate a region of the injection zone of the lens device so that a distal opening of the vent needle is positioned inside the internal chamber of the lens device.

4. The apparatus of claim 3, wherein the vent needle is coupled to a gantry system to achieve desired spatial orientation relative to the lens device.

5. The apparatus of claim 1, wherein the vacuum chamber is sized to contain at least the lens fixture.

6. The apparatus of claim 5, wherein the vacuum pump creates a vacuum inside the vacuum chamber so that the lens device is contained within a negative pressure environment.

7. The apparatus of claim 6, wherein the internal chamber of the lens device and the vacuum chamber are at the same pressure during filling.

8. The apparatus of claim 6, wherein the vacuum chamber is sized to contain the lens fixture and at least a portion of the dispensing system.

9.-11. (canceled)

12. The apparatus of claim 1, further comprising a gantry system coupled to the filling needle, the gantry system configured to movable position the needle in X, Y, and Z spatial orientation.

13. The apparatus of claim 12, wherein the gantry system is configured to advance the filling needle through a lateral side of the lens device within the injection zone parallel to a horizontal plane of the lens device to position the distal opening entirely within the internal chamber of the lens device to be filled by the volume of optical liquid.

14. The apparatus of claim 1, wherein the dispensing system further comprises a needle valve located between the filling needle and the source of optical liquid, the needle valve pneumatically triggered to dispense the volume of optical liquid through the filling needle into the internal chamber.

15. The apparatus of claim 1, wherein the measurement system is configured to measure a radius of curvature, lens power, or sagitta of the lens zone.

16.-17. (canceled)

18. The apparatus of claim 1, wherein the measurement system comprises a non-contact probe positioned on one or more movable stages.

19. The apparatus of claim 18, wherein the non-contact probe is a chromatic confocal sensor or a Shack-Hartmann wavefront sensor.

20. (canceled)

21. The apparatus of claim 1, wherein the measurement system is configured to monitor the lens zone of the lens device simultaneously and in real-time during filling of the internal chamber with the filling needle.

22. The apparatus of claim 1, wherein the lens fixture comprises a recess and a central aperture, wherein the recess is sized and shaped to receive the lens device so that the lens zone of the lens device is positioned over the central aperture.

23.-26. (canceled)

27. The apparatus of claim 1, wherein the dispensing system further comprises a needle valve connected to the dispenser and to the source of optical liquid, the needle valve arranged to control flow of optical liquid into the chamber.

28. The apparatus of claim 27, wherein the needle valve in a closed configuration prevents the optical liquid from flowing through the filling needle and allows for vacuum to build in the vacuum chamber.

29. The apparatus of claim 28, wherein the dispenser is pneumatically triggered to dispense an amount of optical liquid from the source of optical liquid upon opening of the needle valve.

30. A method of delivering an amount of optical liquid into a sealed compartment of an ophthalmic lens device to achieve a refractive power of the ophthalmic device for a patient in need of the ophthalmic lens device while removing residual air from the sealed compartment of the ophthalmic lens device.

31.-39. (canceled)
